(19) United States
(12) Patent Application Publication
(10) Pub. No.: US 2019/0033216 A1
(43) Pub. Date: Jan. 31, 2019

(54) MICROALGAE MONITORING APPARATUS AND MICROALGAE MONITORING METHOD

(71) Applicant: Azbil Corporation, Chiyoda-ku (JP)

(72) Inventor: Kanami IRIE, Chiyoda-ku (JP)

(73) Assignee: Azbil Corporation, Chiyoda-ku (JP)

(21) Appl. No.: 16/060,166

(22) PCT Filed: Oct. 20, 2016
(86) PCT No.: PCT/JP2016/081088

§ 371 Date: Jun. 7, 2018

(30) Foreign Application Priority Data
Dec. 10, 2015 (JP) 2015-241440

(51) Int. Cl. G01N 21/64; G01N 21/05; G01N 21/53; G01N 21/85; C12Q 1/06

(52) U.S. Cl. CPC G01N 21/6486; G01N 21/05; C12Q 1/06; G01N 21/85; G01N 21/53

(57) ABSTRACT

A microalgae observation apparatus includes a flow cell 40 into which a fluid containing microalgae is introduced, an excitation light source 10 configured to irradiate the flow cell 40 with excitation light, a first fluorescence detector 102A configured to detect lipid autofluorescence emitted from lipid of each of the microalgae irradiated with the excitation light, a scattered light detector 105 configured to detect light scattered from each of the microalgae, and a recording section 301 configured to time-sequentially record the intensities of the detected lipid autofluorescence and scattered light. The recording section 301 is included in, for example, a central processing unit (CPU) 300. The lipid of the microalgae is also called oil bodies. The microalgae observation apparatus may further include a display device 401 configured to display changes with time in intensity of autofluorescence emitted from the lipid of the microalgae.

### TECHNICAL FIELD

The present invention relates to environmental technology and to a microalgae monitoring apparatus and a method for rapidly monitoring microalgae.

### BACKGROUND ART

There is a growing interest in using lipid produced by and accumulated in microalgae as a biofuel (see, for example, PTL 1 and NPL 1). For producing a biofuel from microalgae, the microalgae are cultured, and the culture is finished at an appropriate timing. Then, lipid is extracted from the microalgae or the fluid containing the microalgae. The appropriate timing refers to the point that enables the culturing process to produce lipid with a maximum yield. Although it has been reported that chlorophyll, phycoerythrin, and phycocyanin of algae emit autofluorescence (see, for example, NPL 2), there is no report that lipid emits autofluorescence. For examining lipid in microalgae, a method has been proposed in which the lipid in the microalgae is stained with a fluorescent dye, followed by observing the microalgae under a fluorescence microscope. Another method has also been proposed for estimating the lipid content in microalgae according to the color conditions of a suspension containing a large number of microalgae (see, for example, NPL 3).

### CITATION LIST

### Patent Literature

PTL 1: Japanese Unexamined Patent Application Publication No. 2014-174034

### Non Patent Literature

NPL 1: WANG, et al., “Characterization of a green microalga UTEX 2219-4: Effects of photosynthesis and osmotic stress on oil body formation,” Botanical Studies (2011) 53: 305-312.
NPL 2: Saito, et al., “A Method of in situ Measurement for Counting and Sizing of Blue-Green Alga Particles by the Detection of Fluorescent Components at Two Wavelengths” (in Japanese), The Review of Laser Engineering, Vol. 24, issue 4, pp. 59-66
NPL 3: Su et al., “Simultaneous Estimation of Chlorophyll a and Lipid Contents in Microalgae by Three-Color Analysis,” Biotechnology and Bioengineering, Vol. 99, No. 4, Mar. 1, 2008

### SUMMARY OF INVENTION

### Technical Problem

Staining of lipid in microalgae with a fluorescent dye requires human work and also time and effort. It takes time to sample microalgae and measure the microalgae. In addition, fluorescent dyes need to be handled carefully in view of safety and are expensive. Collecting microalgae and measuring the amount of biomass and/or lipid also requires human work, requiring time an effort. In measurements with human work, sampling may vary among methods, and measurement intrinsically includes an error. Furthermore, a microalgae culture tank may be constructed in the nature, such as desert. In this case, it is difficult to often visit the site of the culture to sample and measure the microalgae. Moreover, in the method for estimating the lipid content in microalgae according to the color condition of a suspension containing the microalgae, it is difficult to accurately estimate the lipid content in each microalga. Accordingly, it is an object of the present invention to provide a microalgae monitoring apparatus and a microalgae monitoring method that enable simple, rapid, detailed observation of lipid contained in microalgae.

### Solution to Problem

The present inventors have found, through their intense study, that lipid contained in microalgae emits autofluorescence when the microalgae are irradiated with excitation light.

According to an aspect of the present invention, there is provided a microalgae monitoring apparatus including: (a) a flow cell into which a fluid containing microalgae is introduced; (b) an excitation light source configured to irradiate the flow cell with excitation light; (c) a fluorescence detector configured to detect lipid autofluorescence emitted from lipid of each of the microalgae irradiated with the excitation light; (d) a scattered light detector configured to detect light scattered from each of the microalgae; and (e) a processing unit configured to time-sequentially record the intensities of the detected lipid autofluorescence and scattered light. The lipid autofluorescence may be yellow.

In the microalgae monitoring apparatus, the processing unit may calculate the size of the microalgae from the intensity of the scattered light and calculates the size of the lipid from the intensity of the lipid autofluorescence. The processing unit may calculate the distributions of the size of the microalgae measured within a unit time and the size of the lipid measured within the unit time. The processing unit may shift the unit time for calculating the distributions on a time series. The processing unit may record changes with time in size of the microalgae and in size of the lipid.

In the microalgae monitoring apparatus, the processing unit may calculate the amount and the concentration of the microalgae from the volume of the fluid that has passed through the flow cell within a unit time, the intensity of light scattered from the microalgae within the unit time, and the number of detected signals of the light scattered from the microalgae within the unit time, and calculate the amount and the concentration of the lipid from the volume of the fluid that has passed through the flow cell within a unit time, the intensity of lipid autofluorescence detected within the unit time, and the number of detected signals of lipid autofluorescence emitted within the unit time. The processing unit may record changes with time in amount and concentration of the microalgae and in amount and concentration of the lipid.

The microalgae monitoring apparatus may further include a fluorescence detector configured to detect chloroplast autofluorescence emitted from chloroplasts of each of the microalgae. In the microalgae monitoring apparatus, the processing unit may calculate the size of the microalgae from the intensity of the scattered light, calculate the size of the lipid from the intensity of the lipid autofluorescence, and calculate the size of the chloroplasts from the intensity of the chloroplast autofluorescence. The processing unit may calculate the distributions of the size of the microalgae measured within a unit time, the size of the lipid measured within the unit time, and the size of the chloroplasts measured within the unit time. The processing unit may shift the unit time for calculating the distributions on a time series. The processing unit may record changes with time in size of the microalgae, in size of the lipid, and in size of the chloroplasts.

In the microalgae monitoring apparatus, the processing unit may calculate the amount and the concentration of the microalgae from the volume of the fluid that has passed through the flow cell within a unit time, the intensity of light scattered from the microalgae within the unit time, and the number of detected signals of the light scattered from the microalgae within the unit time, calculate the amount and the concentration of the lipid from the volume of the fluid that has passed through the flow cell within a unit time, the intensity of lipid autofluorescence detected within the unit time, and the number of detected signals of lipid autofluorescence emitted within the unit time, and calculate the amount and the concentration of the chloroplasts from the volume of the fluid that has passed through the flow cell within a unit time, the intensity of chloroplast autofluorescence detected within the unit time, and the number of detected signals of chloroplast autofluorescence emitted within the unit time. The processing unit may record changes with time in amount and concentration of the microalgae, in amount and concentration of the lipid, and in amount and concentration of the chloroplasts.

In the microalgae monitoring apparatus, the flow cell may be connected to a culture vessel in which microalgae are cultured. The fluid containing microalgae may be circulated between the culture vessel and the flow cell. The microalgae monitoring apparatus may further include an output section configured to output calculation results to a culture control device operable to control culture conditions in the culture vessel.

The microalgae monitoring apparatus may further include a display device capable of displaying calculation results.

According to another aspect of the present invention, a method is provided for monitoring microalgae. The method includes: (a) introducing a fluid containing microalgae into a flow cell; (b) irradiating the flow cell with excitation light; (c) detecting lipid autofluorescence emitted from lipid of each of the microalgae irradiated with the excitation light; (d) detecting light scattered from each of the microalgae; and (e) time-sequentially recording the intensities of the detected lipid autofluorescence and scattered light. The lipid autofluorescence may be yellow.

In the microalgae monitoring method may be calculated: the size of the microalgae from the intensity of the scattered light; and the size of the lipid from the intensity of the lipid autofluorescence. The distributions of the size of the microalgae measured within a unit time and the size of the lipid measured within the unit time may be calculated. The unit time for calculating the distributions may be shifted on a time series. Changes with time in size of the microalgae and in size of the lipid may be recorded.

In the microalgae monitoring method may be calculated: the amount and the concentration of the microalgae from the volume of the fluid that has passed through the flow cell within a unit time, the intensity of light scattered from the microalgae within the unit time, and the number of detected signals of the light scattered from the microalgae within the unit time; and the amount and the concentration of the lipid from the volume of the fluid that has passed through the flow cell within a unit time, the intensity of lipid autofluorescence detected within the unit time, and the number of detected signals of lipid autofluorescence emitted within the unit time. Changes with time in amount and concentration of the microalgae and in amount and concentration of the lipid may be recorded.

The microalgae monitoring method may further include detecting chloroplast fluorescence emitted from chloroplasts of each of the microalgae. In the microalgae monitoring method may be calculated: the size of the microalgae from the intensity of the scattered light; the size of the lipid from the intensity of the lipid autofluorescence; and the size of the chloroplasts from the intensity of the chloroplast autofluorescence. The distributions of the size of the microalgae measured within a unit time, the size of the lipid measured within the unit time, and the size of the chloroplasts measured within the unit time may be calculated. The unit time for calculating the distributions may be shifted on a time series. Changes with time in size of the microalgae, in size of the lipid, and in size of the chloroplasts may be recorded.

In the microalgae monitoring method may be calculated: the amount and the concentration of the microalgae from the volume of the fluid that has passed through the flow cell within a unit time, the intensity of light scattered from the microalgae within the unit time, and the number of detected signals of the light scattered from the microalgae within the unit time; the amount and the concentration of the lipid from the volume of the fluid that has passed through the flow cell within a unit time, the intensity of lipid autofluorescence detected within the unit time, and the number of detected signals of lipid autofluorescence emitted within the unit time; and the amount and the concentration of the chloroplasts from the volume of the fluid that has passed through the flow cell within a unit time, the intensity of chloroplast autofluorescence detected within the unit time, and the number of detected signals of chloroplast autofluorescence emitted within the unit time. Changes with time in amount and concentration of the microalgae, in amount and concentration of the lipid, and in amount and concentration of the chloroplasts may be recorded.

In the microalgae monitoring method, the flow cell may be connected to a culture vessel in which microalgae are cultured. The fluid containing microalgae may be circulated between the culture vessel and the flow cell. The microalgae monitoring method may further include outputting calculation results to a culture control device operable to control culture conditions in the culture vessel.

The microalgae monitoring method may further include displaying calculation results.

Also, according to another aspect of the present invention, a method is provided for determining a timing at which a microalgae culture is to be finished. The method includes: (a) introducing a fluid containing microalgae into a flow cell; (b) irradiating the flow cell with excitation light; (c) detecting lipid autofluorescence emitted from lipid of each of the microalgae irradiated with the excitation light; (d) time-sequentially recording the intensity of the detected lipid autofluorescence; and (e) calculating the amount and the concentration of the lipid from the volume of the fluid that has passed through the flow cell within a unit time, the intensity of lipid autofluorescence detected within the unit time, and the number of detected signals of lipid autofluorescence emitted within the unit time; and (f) determining, when the amount and the concentration of the lipid each exceed a predetermined criterion value, that this time is the timing at which the microalgae culture is to be finished. The lipid autofluorescence may be yellow.

In the method for determining the timing of finishing the microalgae culture may be calculated: the size of the microalgae from the intensity of the scattered light; and the size of the lipid from the intensity of the lipid autofluorescence. The distributions of the size of the microalgae measured within a unit time and the size of the lipid measured within the unit time may be calculated. The unit time for calculating the distributions may be shifted on a time series. Changes with time in size of the microalgae and in size of the lipid may be recorded.

In the method for determining the timing of finishing the microalgae culture may be calculated: the amount and the concentration of the microalgae from the volume of the fluid that has passed through the flow cell within a unit time, the intensity of light scattered from the microalgae within the unit time, and the number of detected signals of the light scattered from the microalgae within the unit time; and the amount and the concentration of the lipid from the volume of the fluid that has passed through the flow cell within a unit time, the intensity of lipid autofluorescence detected within the unit time, and the number of detected signals of lipid autofluorescence emitted within the unit time. Changes with time in amount and concentration of the microalgae and in amount and concentration of the lipid may be recorded.

The method for determining the timing of finishing the microalgae culture may further include detecting chloroplast fluorescence emitted from chloroplasts of each of the microalgae. In the method for determining the timing of finishing the microalgae culture may be calculated: the size of the microalgae from the intensity of the scattered light; the size of the lipid from the intensity of the lipid autofluorescence; and the size of the chloroplasts from the intensity of the chloroplast autofluorescence. The distributions of the size of the microalgae measured within a unit time, the size of the lipid measured within the unit time, and the size of the chloroplasts measured within the unit time may be calculated. The unit time for calculating the distributions may be shifted on a time series. Changes with time in size of the microalgae, in size of the lipid, and in size of the chloroplasts may be recorded.

In the method for determining the timing of finishing the microalgae culture may be calculated: the amount and the concentration of the microalgae from the volume of the fluid that has passed through the flow cell within a unit time, the intensity of light scattered from the microalgae within the unit time, and the number of detected signals of the light scattered from the microalgae within the unit time; the amount and the concentration of the lipid from the volume of the fluid that has passed through the flow cell within a unit time, the intensity of lipid autofluorescence detected within the unit time, and the number of detected signals of lipid autofluorescence emitted within the unit time; and the amount and the concentration of the chloroplasts from the volume of the fluid that has passed through the flow cell within a unit time, the intensity of chloroplast autofluorescence detected within the unit time, and the number of detected signals of chloroplast autofluorescence emitted within the unit time. Changes with time in amount and concentration of the microalgae, in amount and concentration of the lipid, and in amount and concentration of the chloroplasts may be recorded.

In the method for determining the timing of finishing the microalgae culture, the flow cell may be connected to a culture vessel in which microalgae are cultured. The fluid containing microalgae may be circulated between the culture vessel and the flow cell. The method for determining the timing of finishing the microalgae culture may further include outputting calculation results to a culture control device operable to control culture conditions in the culture vessel.

The method for determining the timing of finishing the microalgae culture may further include displaying calculation results.

Also, according to another aspect of the present invention, a method is provided for screening microalgae. The method includes: (a) introducing each of a plurality of fluids into a flow cell, the fluids each containing a different kind of microalgae from the microalgae in the other fluids; (b) irradiating the flow cell with excitation light; (c) detecting lipid autofluorescence emitted from lipid of each of the microalgae irradiated with the excitation light; (d) time-sequentially recording the intensity of the detected lipid autofluorescence for each kind of microalgae; (e) calculating the amount and the concentration of the lipid for each kind of microalgae from the volume of the corresponding fluid that has passed through the flow cell within a unit time, the intensity of lipid autofluorescence detected within the unit time, and the number of detected signals of lipid autofluorescence emitted within the unit time; and (f) selecting the kind of microalgae in which the amount and the concentration of the lipid each exceed a predetermined criterion value. The lipid autofluorescence may be yellow.

In the microalgae screening method may be calculated: the size of microalgae from the intensity of scattered light; and the size of lipid from the intensity of lipid autofluorescence. The distributions of the size of the microalgae measured within a unit time and the size of the lipid measured within the unit time may be calculated. The unit time for calculating the distributions may be shifted on a time series. Changes with time in size of the microalgae and in size of the lipid may be recorded.

In the microalgae screening method may be calculated: the amount and the concentration of microalgae from the volume of the fluid that has passed through the flow cell within a unit time, the intensity of light scattered from the microalgae within the unit time, and the number of detected signals of the light scattered from the microalgae within the unit time; and the amount and the concentration of lipid from the volume of the fluid that has passed through the flow cell within a unit time, the intensity of lipid autofluorescence detected within the unit time, and the number of detected signals of lipid autofluorescence emitted within the unit time. Changes with time in amount and concentration of the microalgae and in amount and concentration of the lipid may be recorded.

The microalgae screening method may further include detecting chloroplast fluorescence emitted from chloroplasts of each of the microalgae. In the microalgae screening method may be calculated: the size of microalgae from the intensity of scattered light; the size of lipid from the intensity of lipid autofluorescence; and the size of chloroplasts from the intensity of chloroplast autofluorescence. The distributions of the size of the microalgae measured within a unit time, the size of the lipid measured within the unit time, and the size of the chloroplasts measured within the unit time may be calculated. The unit time for calculating the distributions may be shifted on a time series. Changes with time in size of the microalgae, in size of the lipid, and in size of the chloroplasts may be recorded.

In the microalgae screening method may be calculated: the amount and the concentration of microalgae from the volume of the fluid that has passed through the flow cell within a unit time, the intensity of light scattered from the microalgae within the unit time, and the number of detected signals of the light scattered from the microalgae within the unit time; the amount and the concentration of lipid from the volume of the fluid that has passed through the flow cell within a unit time, the intensity of lipid autofluorescence detected within the unit time, and the number of detected signals of lipid autofluorescence emitted within the unit time; and the amount and the concentration of chloroplasts from the volume of the fluid that has passed through the flow cell within a unit time, the intensity of chloroplast autofluorescence detected within the unit time, and the number of detected signals of chloroplast autofluorescence emitted within the unit time. Changes with time in amount and concentration of the microalgae, in amount and concentration of the lipid, and in amount and concentration of the chloroplasts may be recorded.

In the microalgae screening method, the flow cell may be connected to a culture vessel in which microalgae are cultured. The fluid containing microalgae may be circulated between the culture vessel and the flow cell. The microalgae screening method may further include outputting calculation results to a culture control device operable to control culture conditions in the culture vessel.

The microalgae screening method may further include displaying calculation results.

Also, according to another aspect of the present invention, a method is provided for screening microalgae culture conditions. The method includes: (a) introducing a plurality of fluids into a flow cell, the fluids each containing microalgae being cultured under a condition different from the microalgae in the other fluids (b) irradiating the flow cell with excitation light; (c) detecting lipid autofluorescence emitted from lipid of each of the microalgae irradiated with the excitation light; (d) time-sequentially recording the intensity of the detected lipid autofluorescence for each microalgae culture condition; (e) calculating the amount and the concentration of the lipid for each microalgae culture condition from the volume of the corresponding fluid that has passed through the flow cell within a unit time, the intensity of lipid autofluorescence detected within the unit time, and the number of detected signals of lipid autofluorescence emitted within the unit time; and (f) selecting the culture condition in which the amount and the concentration of the lipid each exceed a predetermined criterion value. The lipid autofluorescence may be yellow.

In the method for screening microalgae culture conditions may be calculated: the size of microalgae from the intensity of scattered light; and the size of lipid from the intensity of lipid autofluorescence. The distributions of the size of the microalgae measured within a unit time and the size of the lipid measured within the unit time may be calculated. The unit time for calculating the distributions may be shifted on a time series. Changes with time in size of the microalgae and in size of the lipid may be recorded.

In the method for screening microalgae culture conditions may be calculated: the amount and the concentration of microalgae from the volume of the fluid that has passed through the flow cell within a unit time, the intensity of light scattered from the microalgae within the unit time, and the number of detected signals of the light scattered from the microalgae within the unit time; and the amount and the concentration of lipid from the volume of the fluid that has passed through the flow cell within a unit time, the intensity of lipid autofluorescence detected within the unit time, and the number of detected signals of lipid autofluorescence emitted within the unit time. Changes with time in amount and concentration of the microalgae and in amount and concentration of the lipid may be recorded.

The method for screening microalgae culture conditions may further include detecting chloroplast fluorescence emitted from chloroplasts of each of the microalgae. In the method for screening microalgae culture conditions may be calculated: the size of microalgae from the intensity of scattered light; the size of lipid from the intensity of lipid autofluorescence; and the size of chloroplasts from the intensity of chloroplast autofluorescence. The distributions of the size of the microalgae measured within a unit time, the size of the lipid measured within the unit time, and the size of the chloroplasts measured within the unit time may be calculated. The unit time for calculating the distributions may be shifted on a time series. Changes with time in size of the microalgae, in size of the lipid, and in size of the chloroplasts may be recorded.

In the method for screening microalgae culture conditions may be calculated: the amount and the concentration of microalgae from the volume of the fluid that has passed through the flow cell within a unit time, the intensity of light scattered from the microalgae within the unit time, and the number of detected signals of the light scattered from the microalgae within the unit time; the amount and the concentration of lipid from the volume of the fluid that has passed through the flow cell within a unit time, the intensity of lipid autofluorescence detected within the unit time, and the number of detected signals of lipid autofluorescence emitted within the unit time; and the amount and the concentration of chloroplasts from the volume of the fluid that has passed through the flow cell within a unit time, the intensity of chloroplast autofluorescence detected within the unit time, and the number of detected signals of chloroplast autofluorescence emitted within the unit time. Changes with time in amount and concentration of the microalgae, in amount and concentration of the lipid, and in amount and concentration of the chloroplasts may be recorded.

In the method for screening microalgae culture conditions, the flow cell may be connected to a culture vessel in which microalgae are cultured. The fluid containing microalgae may be circulated between the culture vessel and the flow cell. The method for screening microalgae culture conditions may further include outputting calculation results to a culture control device operable to control culture conditions in the culture vessel.

The method for screening microalgae culture conditions may further include displaying calculation results.

According to another aspect of the present invention, a method is provided for monitoring environment. The method includes: (a) introducing a fluid containing microalgae into a flow cell; (b) irradiating the flow cell with excitation light; (c) detecting lipid autofluorescence emitted from lipid of each of the microalgae irradiated with the excitation light; (d) detecting chloroplast autofluorescence emitted from chloroplasts of each of the microalgae irradiated with the excitation light; (e) detected light scattered from each of the microalgae; (f) estimating the state of the microalgae from the intensity of the detected lipid autofluorescence, the number of detected signals of lipid autofluorescence emitted within a unit time, the intensity of the detected chloroplast autofluorescence, the number of detected signals of chloroplast autofluorescence emitted within the unit time, the intensity of the detected scattered light, and the number of detected signals of light scattered within the unit time; and (g) estimating the environment of the source of the fluid containing the microalgae from a result of the estimation of the state of the microalgae. The lipid autofluorescence may be yellow.

In the environment monitoring method may be calculated: the size of the microalgae from the intensity of the scattered light; and the size of the lipid from the intensity of the lipid autofluorescence. The distributions of the size of the microalgae measured within a unit time and the size of the lipid measured within the unit time may be calculated. The unit time for calculating the distributions may be shifted on a time series. Changes with time in size of the microalgae and in size of the lipid may be recorded.

In the environment monitoring method may be calculated: the amount and the concentration of the microalgae from the volume of the fluid that has passed through the flow cell within a unit time, the intensity of light scattered from the microalgae within the unit time, and the number of detected signals of the light scattered from the microalgae within the unit time; and the amount and the concentration of the lipid from the volume of the fluid that has passed through the flow cell within a unit time, the intensity of lipid autofluorescence detected within the unit time, and the number of detected signals of lipid autofluorescence emitted within the unit time. Changes with time in amount and concentration of the microalgae and in amount and concentration of the lipid may be recorded.

The environment monitoring method may further include detecting chloroplast fluorescence emitted from chloroplasts of each of the microalgae. In the environment monitoring method may be calculated: the size of the microalgae from the intensity of the scattered light; the size of the lipid from the intensity of the lipid autofluorescence; and the size of the chloroplasts from the intensity of the chloroplast autofluorescence. The distributions of the size of the microalgae measured within a unit time, the size of the lipid measured within the unit time, and the size of the chloroplasts measured within the unit time may be calculated. The unit time for calculating the distributions may be shifted on a time series. Changes with time in size of the microalgae, in size of the lipid, and in size of the chloroplasts may be recorded.

In the environment monitoring method may be calculated: the amount and the concentration of microalgae from the volume of the fluid that has passed through the flow cell within a unit time, the intensity of light scattered from the microalgae within the unit time, and the number of detected signals of the light scattered from the microalgae within the unit time; the amount and the concentration of lipid from the volume of the fluid that has passed through the flow cell within a unit time, the intensity of lipid autofluorescence detected within the unit time, and the number of detected signals of lipid autofluorescence emitted within the unit time; and the amount and the concentration of chloroplasts from the volume of the fluid that has passed through the flow cell within a unit time, the intensity of chloroplast autofluorescence detected within the unit time, and the number of detected signals of chloroplast autofluorescence emitted within the unit time. Changes with time in amount and concentration of the microalgae, in amount and concentration of the lipid, and in amount and concentration of the chloroplasts may be recorded.

In the environment monitoring method, the flow cell may be connected to a culture vessel in which microalgae are cultured. The fluid containing microalgae may be circulated between the culture vessel and the flow cell. The environment monitoring method may further include outputting calculation results to a culture control device operable to control culture conditions in the culture vessel.

The environment monitoring method may further include displaying calculation results.

### Advantageous Effects of Invention

The present invention provides a microalgae monitoring apparatus and a microalgae monitoring method that enable simple, rapid, detailed observation of lipid contained in microalgae.

### BRIEF DESCRIPTION OF DRAWINGS

FIG. 1 is a diagram of a microalgae observation apparatus according to a first embodiment of the present invention.

FIG. 2 is a diagram of a microalga containing lipid and chloroplasts.

FIG. 3 is a representation of an exemplary set of information data stored in a recording device according to the first embodiment of the present invention.

FIG. 4 is a representation of an example of some sets of data stored in a recording device according to the first embodiment of the present invention.

FIG. 5 is a schematic graph showing changes with time, stored in the recording device according to the first embodiment of the present invention, in intensity of light scattered from microalgae, in intensity of autofluorescence emitted from lipid of the microalgae, and in intensity of autofluorescence emitted from chloroplasts of the microalgae.

FIG. 6 is a schematic illustrative representation of changes of a microalga containing lipid and chloroplasts with time.

FIG. 7 is a diagram illustrating a flow cell and a culture vessel of the microalgae observation apparatus according to the first embodiment of the present invention.

FIG. 8 is an exemplary histogram of scattered light intensity in the first embodiment of the present invention.

FIG. 9 is an exemplary histogram of autofluorescence from lipid and autofluorescence from chloroplasts in the first embodiment of the present invention.

FIG. 10 is a diagram illustrating a flow cell and a culture vessel of the microalgae observation apparatus according to the first embodiment of the present invention.

FIG. 11 is an exemplary histogram of scattered light intensity in the first embodiment of the present invention.

FIG. 12 is an exemplary histogram of autofluorescence from lipid and autofluorescence from chloroplasts in the first embodiment of the present invention.

FIG. 13 is a diagram illustrating a flow cell and a culture vessel of the microalgae observation apparatus according to the first embodiment of the present invention.

FIG. 14 is an exemplary histogram of scattered light intensity in the first embodiment of the present invention.

FIG. 15 is an exemplary histogram of autofluorescence from lipid and autofluorescence from chloroplasts in the first embodiment of the present invention.

FIG. 16 is a diagram of a microalgae observation apparatus according to a second embodiment of the present invention.

FIG. 17 is a diagram of a microalga containing lipid and chloroplasts.

FIG. 18 is a diagram of a microalga containing lipid and chloroplasts.

FIG. 19 is a micrograph of chlorella not stained with a fluorescent dye, taken in Reference Example 1.

FIG. 20 is a micrograph of autofluorescence from the chlorella not stained with a fluorescent dye, taken in Reference Example 1.

FIG. 21 shows a micrograph of autofluorescence from the chlorella not stained with a fluorescent dye and an image of the autofluorescence extracted, obtained in Reference Example 1.

FIG. 22 is a superimposed image of the image of extracted autofluorescence put on the micrograph of the chlorella not stained with a fluorescent dye, formed in Reference Example 1.

FIG. 23 is a micrograph of chlorella stained with a fluorescent dye, taken in Reference Example 2.

FIG. 24 is a micrograph of fluorescence from the chlorella stained with a fluorescent dye, taken in Reference Example 2.

FIG. 25 shows a micrograph of fluorescence from the chlorella stained with a fluorescent dye and an image of the autofluorescence extracted, obtained in Reference Example 2.

FIG. 26 is a superimposed image of the image of extracted fluorescence put on the micrograph of the chlorella stained with a fluorescent dye, formed in Reference Example 2.

FIG. 27 is a micrograph of chlorella not stained with a fluorescent dye, taken in Reference Example 3.

FIG. 28 is a micrograph of autofluorescence from the chlorella not stained with a fluorescent dye, taken in Reference Example 3.

FIG. 29 shows a micrograph of autofluorescence from the chlorella not stained with a fluorescent dye and an image of the autofluorescence extracted, obtained in Reference Example 3.

FIG. 30 is a superimposed image of the image of extracted autofluorescence put on the micrograph of the chlorella not stained with a fluorescent dye, formed in Reference Example 3.

FIG. 31 is a micrograph of chlorella stained with a fluorescent dye, taken in Reference Example 4.

FIG. 32 is a micrograph of fluorescence from the chlorella stained with a fluorescent dye, taken in Reference Example 4.

FIG. 33 shows a micrograph of fluorescence from the chlorella stained with a fluorescent dye and an image of the fluorescence extracted, obtained in Reference Example 4.

FIG. 34 is a superimposed image of the image of extracted fluorescence put on the micrograph of the chlorella stained with a fluorescent dye, formed in Reference Example 4.

### DESCRIPTION OF EMBODIMENTS

Some embodiments of the present invention will now be described. It should not be appreciated that the present invention is limited to the description and drawings that are part of the present disclosure. It should be appreciated that various alternations and operations will become apparent to those skilled in the art from the detailed description disclosed herein and that the present invention includes various other embodiments not described herein.

### First Embodiment

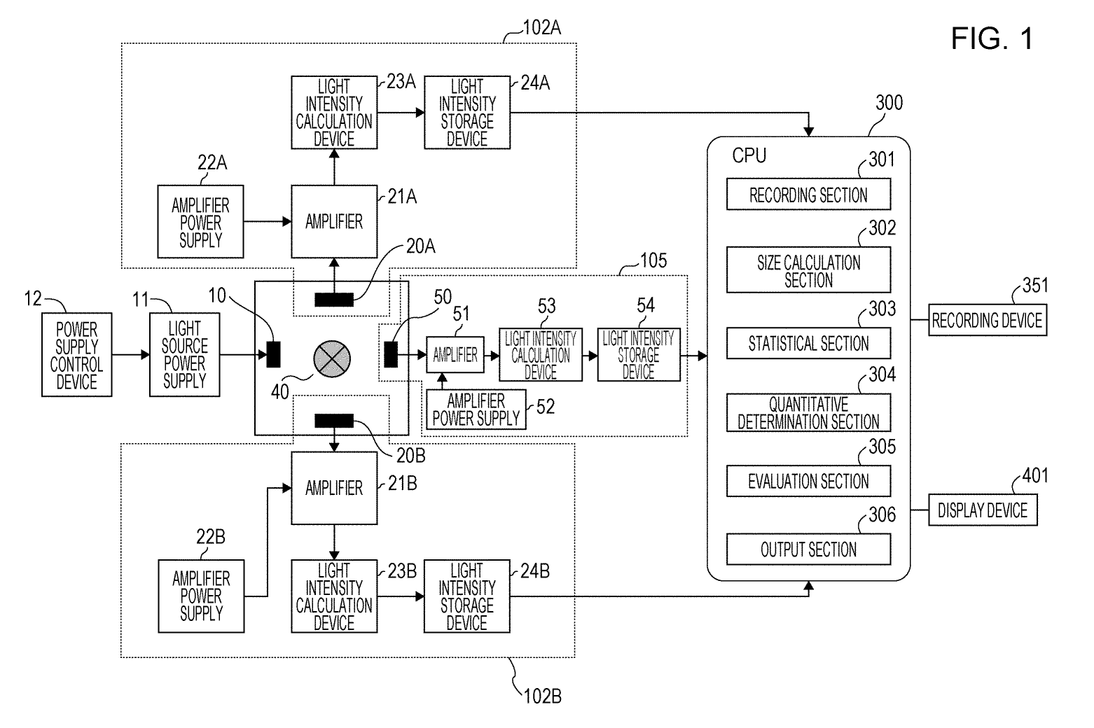

As shown in FIG. 1, a microalgae observation apparatus according to a first embodiment includes a flow cell 40 into which a fluid containing microalgae is introduced, an excitation light source 10 configured to irradiate the flow cell 40 with excitation light, a first fluorescence detector 102A configured to detect autofluorescence emitted from lipid of each of the microalgae irradiated with the excitation light, a scattered light detector 105 configured to detect light scattered from each of the microalgae, and a recording section 301 configured to time-sequentially record the intensities of the detected autofluorescence and scattered light. The recording section 301 may be included in, for example, a central processing unit (CPU) 300. The lipid of the microalgae is also called oil bodies. The fluid that flows in the flow cell 40 may be liquid or gas. In the following description, an embodiment using liquid as the fluid will be described by way of example. The lipid may be secreted out of microalgae and present in the fluid.

The excitation light source 10 emits excitation light in a wide range of wavelengths to the fluid flowing in the flow cell 40. The excitation light source 10 may be, for example, a light-emitting diode (LED) or a laser. The excitation light may be, for example, blue light having a wavelength of 450 nm to 495 nm. The wavelength and the color of the excitation light are however not limited to these. The excitation light may be visible light other than blue light, such as purple light, or ultraviolet light. The wavelength range of the excitation light may be set by using a filter such as a band-pass filter. The excitation light is focused on a point, for example, within the flow cell 40. The excitation light source 10 is connected to a light source driving power supply 11 configured to supply electric power to the excitation light source 10. The light source driving power supply 11 is connected to a power supply control device 12 configured to control the electric power to be supplied to the excitation light source 10.

The flow cell 40 is transparent to the excitation light and may be made of, for example, quartz. The flow cell 40 has an inner diameter that allows microalgae to flow therein approximately one by one. The flow cell 40 may be in a shape of a round tube or a rectangular tube. The flow cell 40 may be connected to, for example, a culture vessel in which microalgae are cultured. Alternatively, a liquid containing microalgae under cultivation in a culture vessel may be introduced into the flow cell 40 in regular intervals. The fluid containing microalgae may be circulated between the culture vessel and the flow cell 40. A small amount of fluid containing microalgae sampled from the culture vessel may be intermittently introduced into the flow cell 40. The fluid flowing within the flow cell 40 passes across the beam of excitation light.

Microalgae are unicellular algae of, for example, several micrometers to several tens of micrometers in size. Microalgae may be called phytoplankton. For example, microalgae produce hydrocarbons. Exemplary microalgae include Botryococcus braunii, Aurantiochytrium, Pseudochoricystis ellipsoidea, Scenedesmus, Desmodesmus, chlorella, Dunaliella, Arthrospira, Spirulina, Euglena, Nannochloropsis, Haematococcus, and Microcystis aeruginosa.

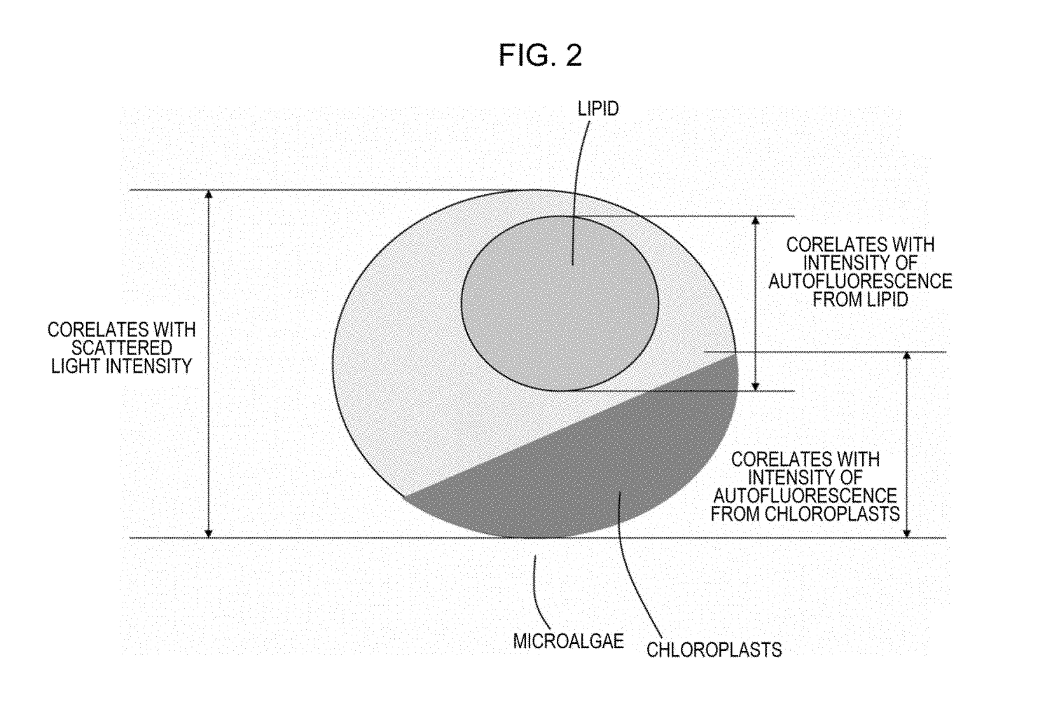

If the fluid flowing in the flow cell 40 contains microalgae, the lipid of the microalgae irradiated with excitation light emits yellow autofluorescence having wavelengths of about 540 nm to 620 nm. The peak wavelength of the lipid autofluorescence is generally 570 nm to 590 nm. As shown in FIG. 2, the intensity of the autofluorescence emitted from lipid is reflective of the size of the lipid contained in a microalga. The chloroplasts of the microalga irradiated with excitation light emit red autofluorescence in a wavelength range of about 650 nm to 730 nm. The peak wavelength of the chloroplast autofluorescence is generally 680 nm to 700 nm. The intensity of the autofluorescence emitted from chloroplasts is reflective of the size of the chloroplasts contained in a microalga. The excitation wavelength for lipid autofluorescence may be the same as the excitation wavelength for chloroplast autofluorescence. Microalgae irradiated with excitation light cause Mie scattering to produce scatted light. The intensity of the scattered light is reflective of the total size of one microalga.

The term “size” used herein refers to, for example, diameter, area, or volume. For example, the microalga, the region defined by the lipid in the microalga, and the chloroplast each have a shape approximated by a particle, the size may be referred to as the particle size.

The wavelength of the autofluorescences is the value when the irradiation is performed with excitation light in a wavelength range of 460 nm to 495 nm through an absorption filter that absorbs light having a wavelength of less than 510 nm and transmits light having a wavelength of 510 nm or more, and may vary depending on conditions. However, the wavelength band of the lipid autofluorescence lies in a shorter region than the wavelength band of the chloroplast autofluorescence, and this relationship is maintained.

As shown in FIG. 1, the first fluorescence detector 102A configured to detect autofluorescence emitted from the lipid of the microalgae includes a first light-receiving element 20A configured to receive autofluorescence emitted from the lipid of the microalgae. A filter, such as an absorption filter, may be disposed for setting the wavelength band of light to be received by the first light-receiving element 20A upstream of the first light-receiving element 20A. Exemplary elements that can be used as the first light-receiving element 20A include a solid-state image pickup device, such as a charge coupled device (CCD) image sensor; an internal photoelectric effect (photovoltaic effect) optical sensor, such as a photodiode; and an external photoelectric effect optical sensor, such as a photomultiplier tube. On receiving the autofluorescence emitted from the lipid, the first light-receiving element 20A converts the optical energy into an electrical energy. The first light-receiving element 20A is connected to an amplifier 21A that amplifies the current generated from the first light-receiving element 20A. The amplifier 21A is connected to an amplifier power supply 22A that supplies power to the amplifier 21A.

The amplifier 21A is connected to a light intensity calculation device 23A that receives the current amplified by the amplifier 21A and calculates the intensity of the autofluorescence emitted from the lipid and received by the first light-receiving element 20A. The light intensity calculation device 23A calculates the intensity of the autofluorescence emitted from the lipid based on, for example, the area of the spectrum of detected autofluorescence. An image analysis software program may be used to calculate the intensity of lipid autofluorescence in the light intensity calculation device 23A. Alternatively, the light intensity calculation device 23A may calculate the intensity of autofluorescence emitted from the lipid based on the magnitude of the electrical signal generated from the first light-receiving element 20A. The light intensity calculation device 23A is connected to a light intensity storage device 24A that stores the intensity of autofluorescence emitted from the lipid, calculated by the light intensity calculation device 23A.

The microalgae observation apparatus according to the first embodiment may further include a second fluorescence detector 102B configured to detect autofluorescence emitted from the chloroplasts of the microalgae. The second fluorescence detector 102B includes a second light-receiving element 20B configured to receive autofluorescence emitted from the chloroplasts of the microalgae. A filter, such as an absorption filter, may be disposed for setting the wavelength band of light to be received by the second light-receiving element 20B upstream of the second light-receiving element 20B. Exemplary elements that can be used as the second light-receiving element 20B include a solid-state image pickup device, such as a charge coupled device (CCD) image sensor; an internal photoelectric effect (photovoltaic effect) optical sensor, such as a photodiode; and an external photoelectric effect optical sensor, such as a photomultiplier tube. On receiving the autofluorescence emitted from the chloroplasts, the second light-receiving element 20B converts the optical energy into an electrical energy. The second light-receiving element 20B is connected to an amplifier 21B that amplifies the current generated from the second light-receiving element 20B. The amplifier 21B is connected to an amplifier power supply 22B that supplies power to the amplifier 21B.

The amplifier 21B is connected to a light intensity calculation device 23B that receives the current amplified by the amplifier 21B and calculates the intensity of the autofluorescence emitted from the chloroplasts and received by the second light-receiving element 20B. The light intensity calculation device 23B calculates the intensity of the autofluorescence emitted from the chloroplasts based on, for example, the area of the spectrum of detected autofluorescence. An image analysis software program may be used to calculate the intensity of autofluorescence emitted from the chloroplasts in the light intensity calculation device 23B. Alternatively, the light intensity calculation device 23B may calculate the intensity of autofluorescence emitted from the chloroplasts based on the magnitude of the electrical signal generated from the second light-receiving element 20B. The light intensity calculation device 23B is connected to a light intensity storage device 24B that stores the intensity of autofluorescence emitted from the chloroplasts, calculated by the light intensity calculation device 23B.

The scattered light detector 105 includes a scattered light-receiving element 50 configured to receive scattered light. Exemplary elements that can be used as the scattered light-receiving element 50 include a solid-state image pickup device, such as a charge coupled device (CCD) image sensor; an internal photoelectric effect (photovoltaic effect) optical sensor, such as a photodiode; and an external photoelectric effect optical sensor, such as a photomultiplier tube. On receiving light, the scattered light-receiving element 50 converts the optical energy into an electrical energy. The scattered light-receiving element 50 is connected to an amplifier 51 that amplifies the current generated from the scattered light-receiving element 50. The amplifier 51 is connected to an amplifier power supply 52 that supplies power to the amplifier 51.

The amplifier 51 is connected to a light intensity calculation device 53 that receives the current amplified by the amplifier 51 and calculates the intensity of the scattered light received by the scattered light-receiving element 50. The light intensity calculation device 53 calculates the intensity of the scattered light based on, for example, the area of the spectrum of detected scattered light. An image analysis software program may be used to calculate the intensity of scattered light in the light intensity calculation device 53. Alternatively, the light intensity calculation device 53 may calculate the intensity of scattered light based on the magnitude of the electrical signal generated from the scattered light-receiving element 50. The light intensity calculation device 53 is connected to a light intensity storage device 54 that stores the intensity of scattered light calculated by the light intensity calculation device 53.

As liquid flows in the flow cell 40, the excitation light source 10 emits excitation light, and the first and second fluorescence detectors 102A and 102B measure the intensity of autofluorescence emitted from the lipid of the microalgae and the intensity of autofluorescence emitted from the chloroplasts of the microalgae, respectively. The intensities are then stored in the respective light intensity storage devices 24A and 24B. Also, the scattered light detector 105 measures scattered light from the microalgae, and the intensity of the scattered light is stored in the light intensity storage device 54. The simultaneously detected autofluorescences in two wavelength bands and the scattered light can be considered to be those derived from identical individuals. Also, if at least scattered light and chloroplast autofluorescence are simultaneously detected, it can be believed that a single microalga has moved across the beam of excitation light. Accordingly, the number of microalgae that have passed through the flow cell 40 can be estimated from the number of times of simultaneous detection of scattered light, lipid autofluorescence and chloroplast autofluorescence.

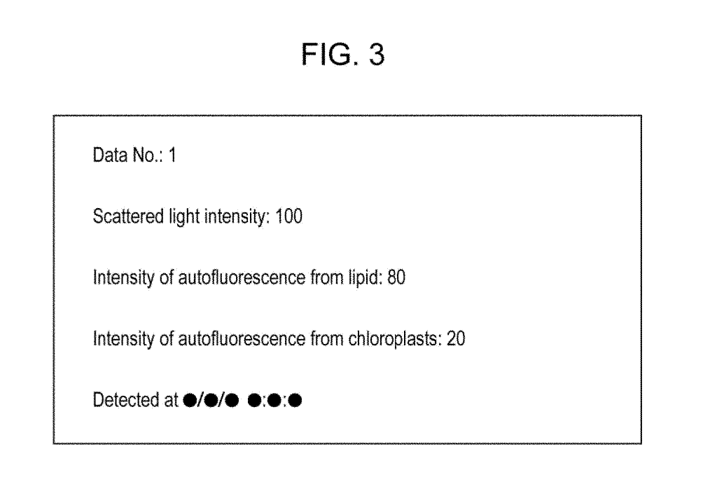

The recording section 301 reads the intensity of the autofluorescence emitted from the lipid of microalgae, and the intensity of the autofluorescence emitted from the chloroplasts of the microalgae from the light intensity storage devices 24A and 24B. The recording section 301 also reads the intensity of scattered light from the microalga from the light intensity storage 54. Furthermore, the recording section 301 adds time data, such as detection date and time, to information including the intensity of scattered light from a single microalga, the intensity of the autofluorescence emitted from the lipid of the microalga, and the intensity of the autofluorescence emitted from the chloroplasts of the microalga, as shown in FIG. 3, and the information is stored in the recording device 351 connected to the CPU 300 shown in FIG. 1.

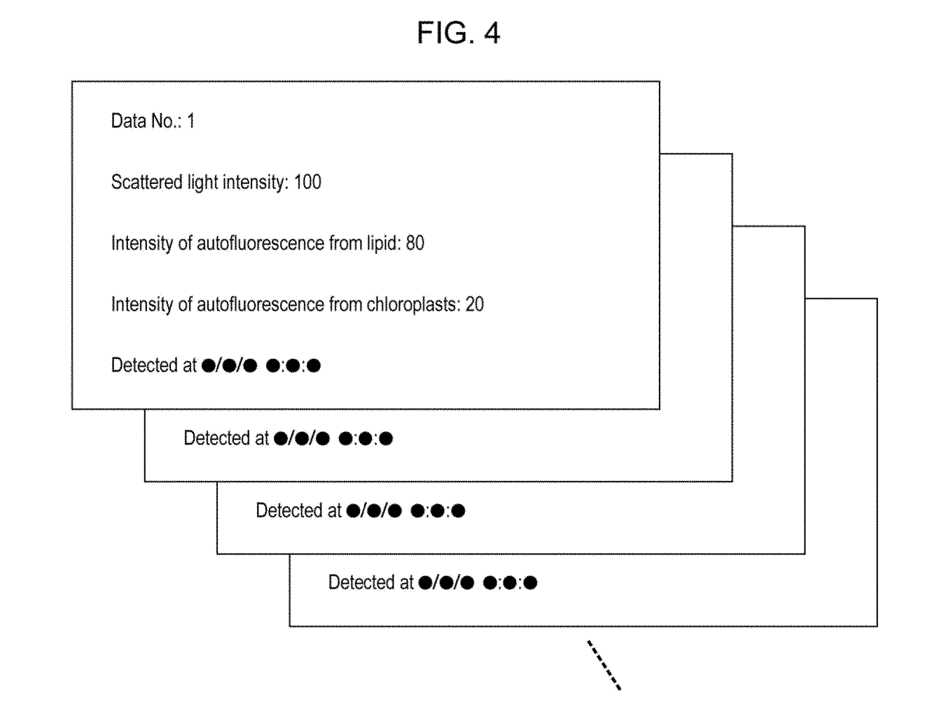

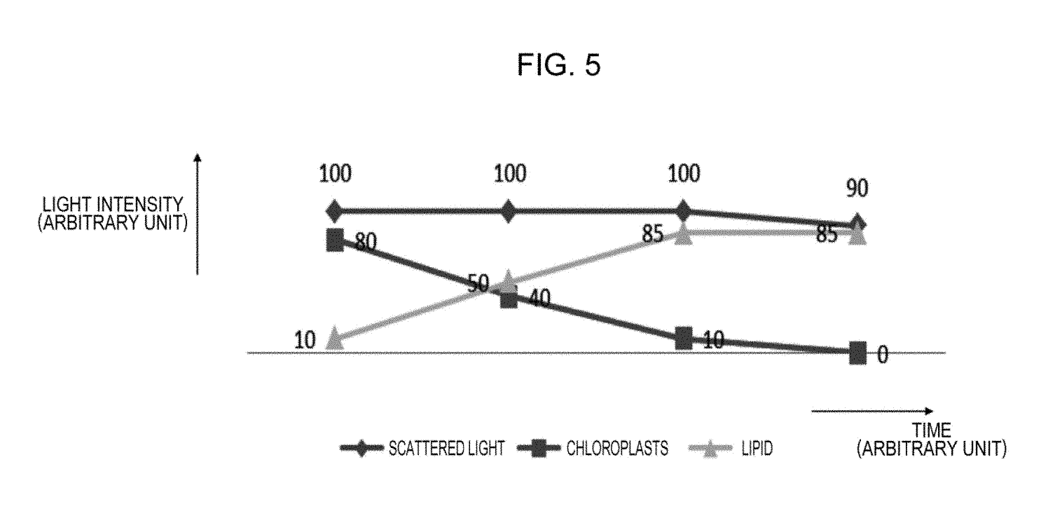

For example, the information including the intensity of scattered light from the microalgae, the intensity of autofluorescence from the lipid of the microalgae, and the intensity of autofluorescence from the chloroplasts of the microalgae is accumulated in the recording device 351 as shown in FIG. 4 by repeating the measurements of the intensity of scattered light from microalgae, the intensity of autofluorescence from the lipid of the microalga, and the intensity of autofluorescence from the chloroplasts of the microalgae for a certain period of time. The recording device 351 thus records the changes with time in intensity of scatted light from the microalgae, the changes with time in intensity of autofluorescence from the lipid of the microalgae, and the changes with time in intensity of autofluorescence from the chloroplasts of the microalgae, as shown in FIG. 5.

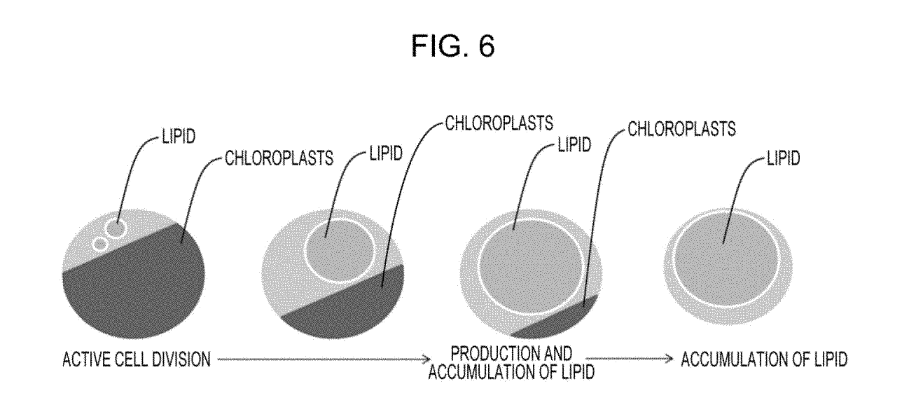

The microalgae undergo active cell division in the early stage of a culture, as shown in, for example, FIG. 6. In this stage, lipid accounts for a small part of a microalga, while chloroplasts account for a large part of the microalgae. As a time elapses for the culture, the frequency of cell division decreases, and the lipid is increasingly produced in the microalga, thus being accumulated in the microalga. Thus, the sizes of the lipid and the chloroplasts relative to the size of the microalga vary depending on the state of the microalga.

As described above, the intensity of scattered light from microalgae is reflective of the total size of one microalga; the intensity of autofluorescence emitted from the lipid of the microalgae is reflective of the size of the lipid in the microalga; and the intensity of autofluorescence emitted from the chloroplasts of the microalgae is reflective of the size of the chloroplasts in the microalga. Thus, by recording the changes with time in intensity of scattered light from microalgae, the changes with time in intensity of autofluorescence emitted from the lipid of the microalgae, and the changes with time in intensity of autofluorescence emitted from the chloroplasts of the microalgae, the changes with time in size of the microalgae, the changes with time in the lipid in the microalgae, and the changes with time in size of the chloroplasts in the microalgae can be estimated. Also, since the sizes of the lipid and the chloroplasts relative to the size of microalgae vary depending on the state of the microalgae, as described above, the state of microalgae can be estimated from the changes with time in size of the microalgae, in size of lipid, and in size of chloroplasts.

The CPU 300 may further include a size calculation section 302. The size calculation section 302 calculates the size of microalgae based on the scattered light from the microalgae. The size calculation section 302 may calculate the size of microalgae based on a relationship previously obtained between the intensity of scattered light and the size of the microalgae.

Also, the size calculation section 302 calculates the size of lipid in microalgae based on the intensity of autofluorescence emitted from the lipid. The size calculation section 302 may calculate the size of lipid based on a relationship previously obtained between the intensity of lipid autofluorescence and the size of the lipid.

Also, the size calculation section 302 calculates the size of chloroplasts in microalgae based on the intensity of autofluorescence emitted from the chloroplasts. The size calculation section 302 may calculate the size of lipid based on a relationship previously obtained between the intensity of autofluorescence from chloroplasts and the size of the chloroplasts.

The recording section 301 may record changes with time in the sizes of microalgae, lipid and chloroplasts that are calculated by the size calculation section 302 in the recording device 351.

The CPU 300 may further include a statistical section 303. The statistical section 303 statistically analyzes the size of microalgae, the size of lipid and the size of chloroplasts that have been measured by introducing microalgae into the flow cell 40 within a predetermine unit time. For example, the statistical section 303 calculates the distribution of the size of microalgae, the distribution of the size of lipid and the distribution of the size of chloroplasts that have been measured by introducing microalgae into the flow cell 40 within a predetermined unit time. The statistical section 303 may prepare a histogram representing the distributions. The term unit time used herein refers to an arbitrarily determine period of time and defines the population for calculating the distributions.

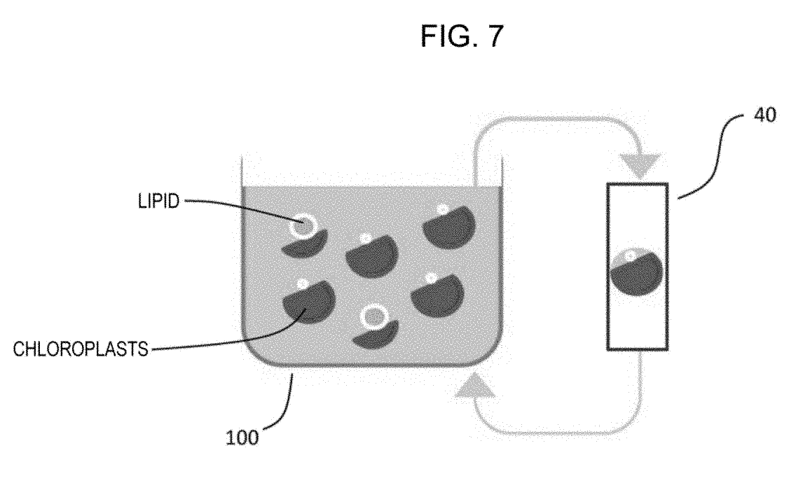

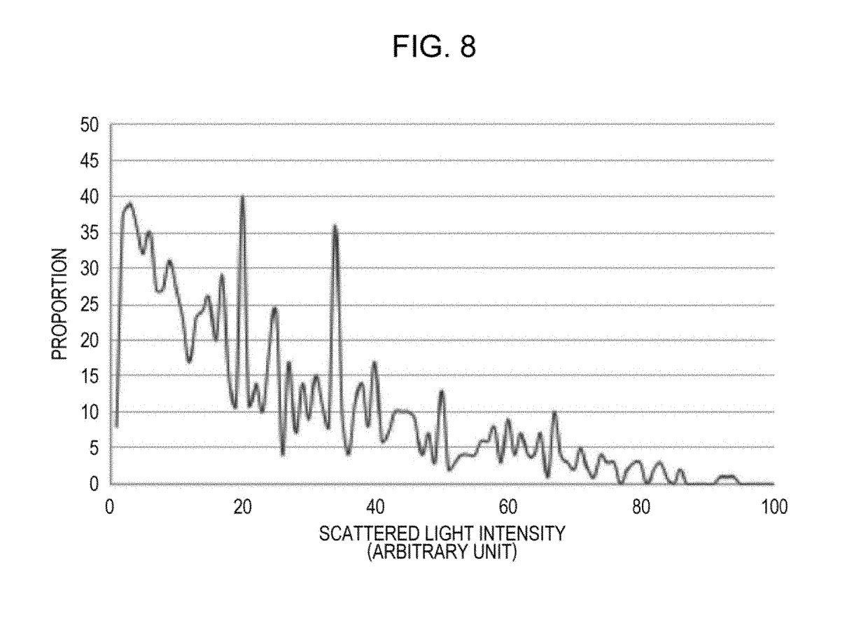

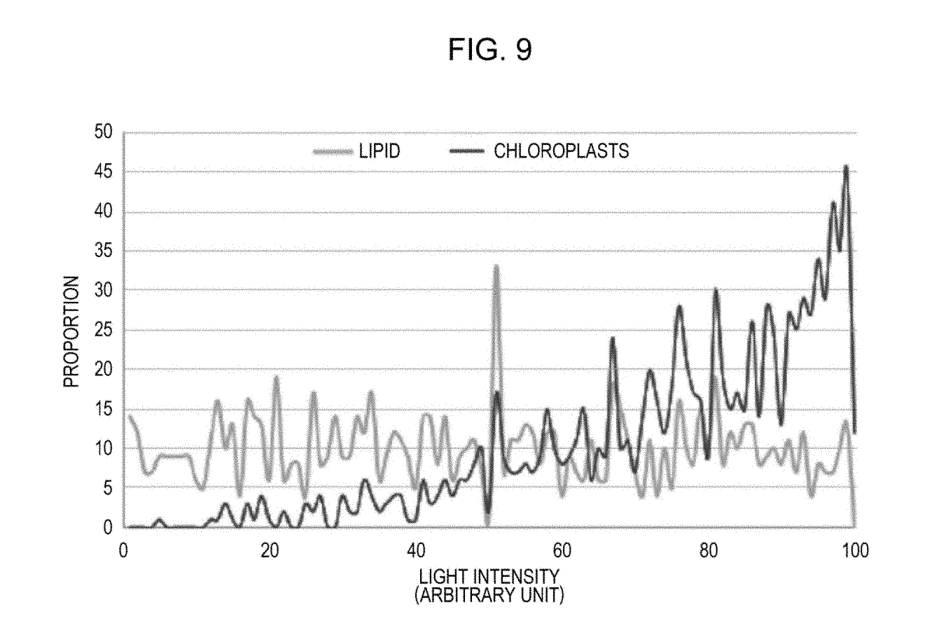

If the intensity of scattered light, the intensity of autofluorescence from lipid, and the intensity of autofluorescence from chloroplasts are measured for each microalga by circulating microalgae undergoing active cell division between a culture vessel 100 and the flow cell 40, as shown in, for example, FIG. 7, a histogram is obtained in which the distribution of scattered light intensity representing the size of the microalga, is biased toward the weaker side of the intensity, as shown in FIG. 8. Also, another histogram is obtained in which the distribution of lipid autofluorescence intensity representing the size of lipid is constant while the distribution of chloroplast autofluorescence intensity representing the size of chloroplasts is biased toward the stronger side of the intensity, as shown in FIG. 9. Thus, the histogram shown in FIG. 8 suggests that the microalgae being cultured in the culture vessel 100 are small. Also, the histogram shown in FIG. 9 suggests that the microalgae being cultured in the culture vessel 100 contain a large amount of chloroplasts.

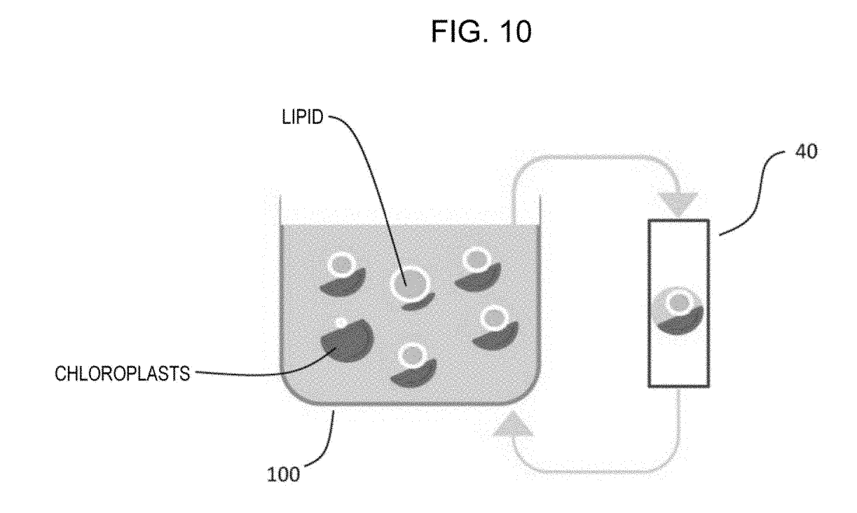

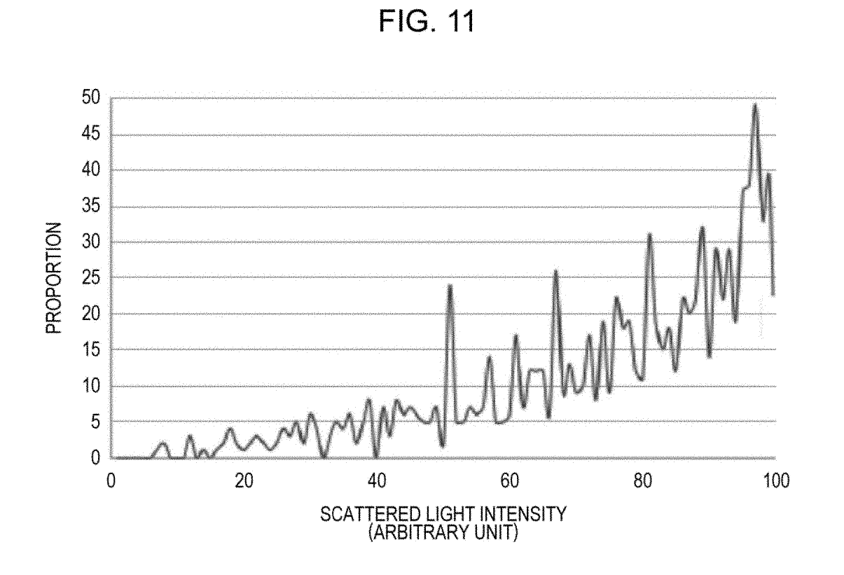

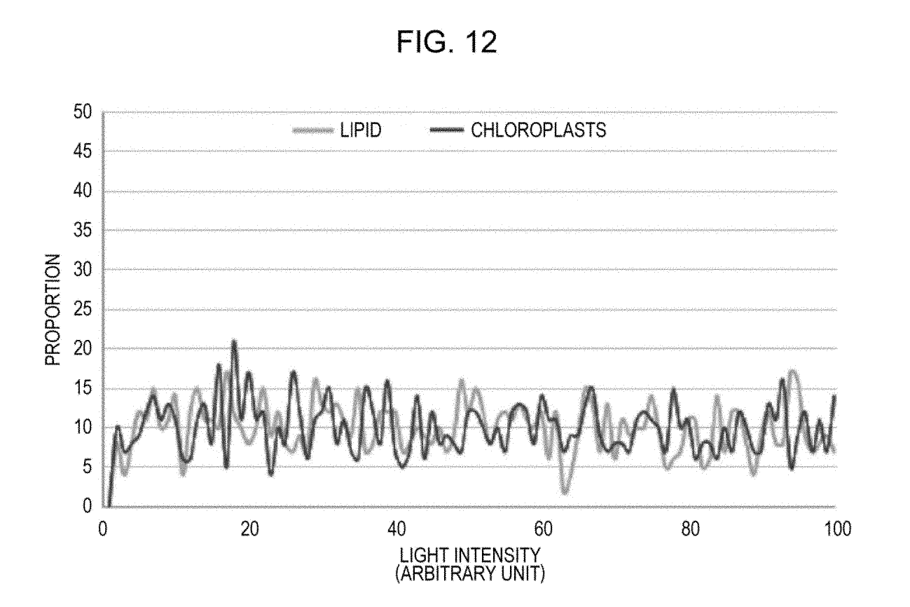

For example, if the intensity of scattered light, the intensity of autofluorescence emitted from lipid, and the intensity of autofluorescence emitted from chloroplasts are measured for each microalga by circulating, between the culture vessel 100 and the flow cell 40, microalgae in a stage in which the amount of lipid produced in the microalgae is almost the same as the amount of chloroplasts in the microalgae, as shown in FIG. 10, a histogram is obtained in which the distribution of scattered light intensity representing the size of the microalgae is biased toward the stronger side of the intensity, as shown in FIG. 11. Also, another histogram is obtained in which both the distribution of lipid autofluorescence intensity representing the size of lipid, and the distribution of chloroplast autofluorescence intensity representing the size of chloroplasts are constant, as shown in FIG. 12. Thus, the histogram shown in FIG. 11 suggests that the microalgae being cultured in the culture vessel 100 are large. Also, the histogram shown in FIG. 10 suggests that the amount of lipid produced in the microalgae being cultured in the culture vessel 100 is almost the same as the amount of chloroplasts in the microalgae.

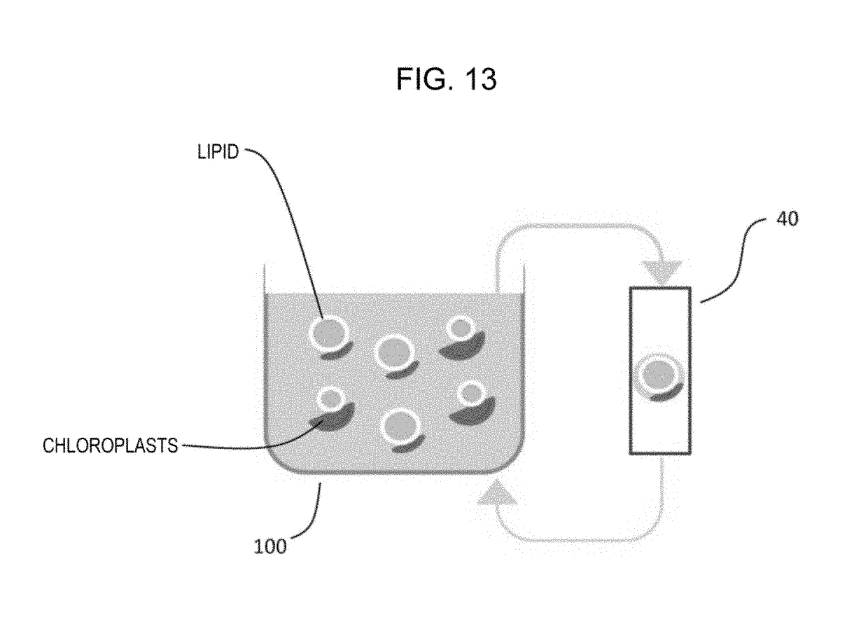

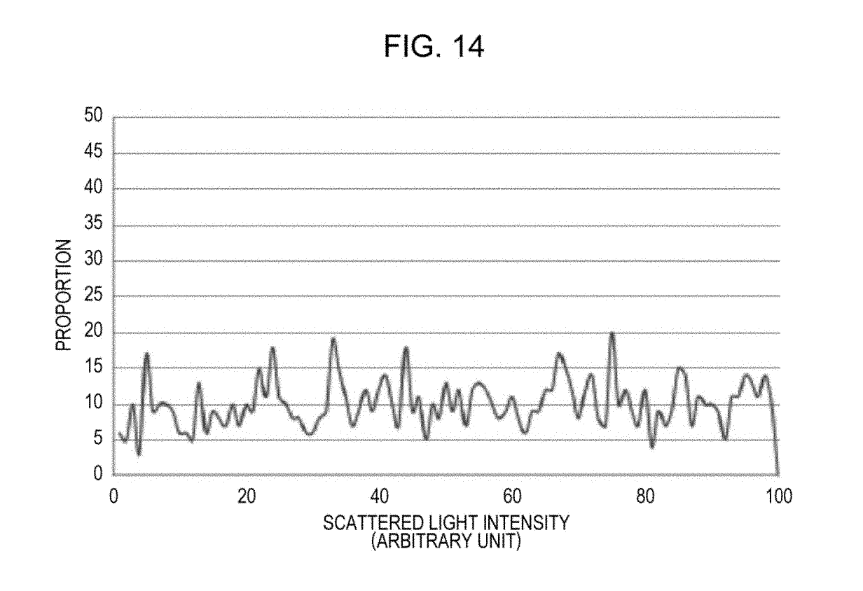

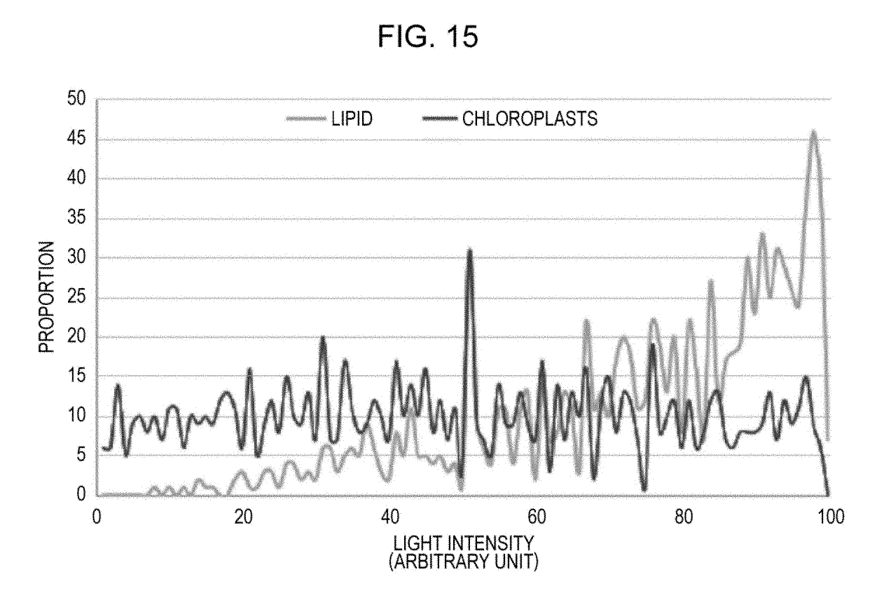

Also, for example, if the intensity of scattered light, the intensity of autofluorescence emitted from lipid, and the intensity of autofluorescence emitted from chloroplasts are measured for each microalga by circulating, between the culture vessel 100 and the flow cell 40, microalgae in a stage in which the amount of lipid produced in the microalgae is larger than the amount of the chloroplasts in the microalgae, as shown in FIG. 13, a histogram is obtained in which the distribution of scattered light intensity representing the size of the microalgae is constant, as shown in FIG. 14. Also, the distribution of lipid autofluorescence intensity representing the size of lipid is biased toward the stronger side of the intensity, as shown in FIG. 15. Thus, the histogram shown in FIG. 14 suggests that the distribution of the size of microalgae being cultured in the culture vessel 100 is constant. Also, the histogram shown in FIG. 15 suggests that the amount of lipid produced in the microalgae being cultured in the culture vessel 100 is large.

The statistical section 303 may prepare a plurality of histograms along a time series, as shown in FIGS. 8, 9, 11, 12, 14, and 15, by shifting a unit time on the time series.

The statistical section 303 may prepare a plurality of histograms along a time series and analyze changes in dispersion with time by superimposing the histograms thus accumulated. The changes of the histograms with time may show the conditions of the microalgae being cultured in the culture vessel.

The recording section 301 may record changes with time of the size distributions of microalgae, lipid and chloroplasts that are calculated by the statistical section 303 in the recording device 351.

The CPU 300 shown in FIG. 1 may further include a quantitative determination section 304. The quantitative determination section 304 calculates the amount and the concentration of microalgae from the volume of the fluid that has passed through the flow cell 40 within a unit time, the intensity of light scattered from microalgae within the unit time, and the number of detected signals of the light scattered from the microalgae within the unit time. For example, the quantitative determination section 304 calculates the integral of the relationship between the intensity of detected signals of light scattered from microalgae within the unit time, plotted on the vertical axis, and the number of the detected signals, plotted on the horizontal axis, as the amount of microalgae. Also, the quantitative determination section 304 calculates the concentration of microalgae in a unit volume of fluid by dividing the amount of microalgae by the volume of the fluid that has passed through the flow cell 40. For example, the quantitative determination section 304 calculates the concentration of microalgae by dividing the number of detected signals of light scattered from microalgae within a unit time by the volume of the fluid that has passed through the flow cell 40 within the unit time.

Furthermore, the quantitative determination section 304 calculates the amount and the concentration of lipid from the volume of the fluid that has passed through the flow cell 40 within a unit time, the intensity of lipid autofluorescence detected within the unit time, and the number of detected signals of lipid autofluorescence emitted within the unit time. For example, the quantitative determination section 304 calculates the integral of the relationship between the intensity of detected signals of lipid autofluorescence emitted within a unit time, plotted on the vertical axis, and the number of the detected signals, plotted on the horizontal axis, as the amount of lipid. Also, the quantitative determination section 304 calculates the concentration of lipid in a unit volume of fluid by dividing the amount of lipid by the volume of the fluid that has passed through the flow cell 40.

The quantitative determination section 304 also calculates the amount of lipid per unit amount of microalgae by dividing the amount of lipid by the amount of microalgae. The quantitative determination section 304 also calculates the concentration of lipid per unit concentration of microalgae by dividing the concentration of lipid by the concentration of microalgae.

Furthermore, the quantitative determination section 304 may calculate, for example, the concentration of microalgae containing a certain amount or more of lipid by dividing the number of signals of lipid autofluorescence detected within a unit time and having an intensity higher than or equal to a certain value by the volume of the fluid that has passed through the flow cell 40 within the unit time.

The quantitative determination section 304 also calculates the amount and the concentration of chloroplasts from the volume of the fluid that has passed through the flow cell 40 within a unit time, the intensity of chloroplast autofluorescence detected within the unit time, and the number of detected signals of chloroplast autofluorescence emitted within the unit time. For example, the quantitative determination section 304 calculates the integral of the relationship between the intensity of detected signals of chloroplast autofluorescence emitted within a unit time, plotted on the vertical axis, and the number of the detected signals, plotted on the horizontal axis, as the amount of chloroplasts. Also, the quantitative determination section 304 calculates the concentration of chloroplasts in a unit volume of fluid by dividing the amount of chloroplasts by the volume of the fluid that has passed through the flow cell 40.

Also, for example, the quantitative determination section 304 calculates the amount of chloroplasts per unit amount of microalgae by dividing the amount of chloroplasts by the amount of microalgae. The quantitative determination section 304 also calculates, for example, the concentration of chloroplasts per unit concentration of microalgae by dividing the concentration of chloroplasts by the concentration of microalgae.

Furthermore, the quantitative determination section 304 may calculate, for example, the concentration of microalgae containing a certain amount or more of chloroplasts by dividing the number of signals of chloroplast autofluorescence detected within a unit time and having an intensity higher than or equal to a certain value by the volume of the fluid that has passed through the flow cell 40 within the unit time.

The recording section 301 may store the changes with time in the amounts and concentrations of microalgae, lipid and chloroplasts that are calculated by the quantitative determination section 304 in the recording device 351.

The CPU 300 shown in FIG. 1 may further include an evaluation section 305. The evaluation section 305 estimates the state of microalgae from the changes with time in intensity of autofluorescence emitted from the lipid of the microalgae. For example, when the distribution of intensity of autofluorescence emitted from the lipid of microalgae exceeds a predetermined criterion value, the evaluation section 305 determines that this is the timing at which the microalgae culture is to be finished.

Alternatively, at that time, the evaluation section 305 determines that the microalgae are in a state suitable to extract the lipid and that it is the timing at which the lipid is to be extracted from the microalgae. The predetermined criterion value may be arbitrarily set according to the kind of microalgae, the culture conditions, the use of lipid to be extracted, and the like. It may be advantageous to collect microalgae from the culture vessel after the intensity of autofluorescence emitted from lipid exceeds a predetermined criterion value and to extract the lipid from the microalgae.

Alternatively, when the amount and the concentration of lipid each exceed a predetermined criterion value, the evaluation section 305 may determine that this is the timing at which the microalgae culture is to be finished, or determine that the microalgae are in a state suitable to extract the lipid and that it is the timing at which the lipid is to be extracted from the microalgae.

The CPU 300 shown in FIG. 1 is connected to a display device 401. The display device 401 displays, for example, the changes with time, stored in the recording device 351, in intensity of light scattered from microalgae, in intensity of autofluorescence emitted from the lipid of the microalgae, and in intensity of autofluorescence emitted from the chloroplasts of the microalgae. The display device 401 also displays the changes with time, stored in the recording device 351, in size of microalgae, in size of lipid, and in size of chloroplasts. Furthermore, the display device 401 displays the changes with time, stored in the recording device 351, of the size distributions of lipid and chloroplasts.

The CPU 300 may further include an output section 306 that outputs calculation results of the size calculation section 302, the statistical section 303, the quantitative determination section 304, and the evaluation section 305 to a culture control device operable to control the culture conditions of the culture vessel connected to the flow cell 40.

The above-described microalgae observation apparatus according to the first embodiment can observe changes with time of the lipid contained in microalgae without previous fluorescent dye staining. For example, if a large amount of microalgae is cultured, it is not easy to stain all the microalgae with a fluorescent dye. However, the microalgae observation apparatus according to the first embodiment enables the lipid contained in microalgae to be time-sequentially observed by continuously introducing the microalgae into a flow cell.

It should be noted that while it has been reported that chlorophyll, phycoerythrin, and phycocyanin, which are kinds of algae, emit autofluorescence, there is no report that lipid emits autofluorescence. This is probably because autofluorescence from lipid has not been noticed or known since lipid is generally examined by being stained with a fluorescent dye.

In recent years, it has been attempted to use lipid contained in microalgae as biofuel, pharmaceuticals, cosmetics, supplements, or the like. The amount of lipid in microalgae varies depending on culture conditions and other environmental conditions, and the proportion in size of the lipid to the total size of the microalgae is not constant. If lipid of microalgae being cultured in a culture vessel is used, however, it is advantageous that the proportion of the size of lipid in each microalga to the size of the corresponding microalga be large.

The microalgae observation apparatus according to the first embodiment enables changes with time of the proportion of the size of lipid to the size of microalgae to be obtained by observing the changes with time in intensity of autofluorescence emitted from the lipid. Thus, this apparatus enables plural kinds of microalgae to be screened to select a kind of microalgae containing a large amount of lipid. It should be noted that the phrase “plural kinds of microalgae” used herein include microalgae derived from a plurality of different strains and microalgae into which a plurality of different genes are respectively introduced, even if they are academically considered to be the same.

A method for screening microalgae may include, for example: introducing each of a plurality of fluids into a flow cell 40, the fluids each containing a different kind of microalgae from the microalgae in the other fluids; irradiating the flow cell 40 with excitation light; detecting autofluorescence emitted from lipid of each of the microalgae irradiated with the excitation light; allowing the recording section 301 to time-sequentially record the intensity of the detected lipid autofluorescence for each kind of microalgae in the recording device 351; allowing the quantitative determination section 304 to calculate the amount and the concentration of the lipid for each kind of microalgae from the volume of the corresponding fluid that has passed through the flow cell 40 within a unit time, the intensity of lipid autofluorescence detected within the unit time, and the number of detected signals of lipid autofluorescence emitted within the unit time; and selecting the kind of microalgae in which the amount and the concentration of the lipid each exceed a predetermined criterion value. The predetermined criterion values are appropriately set.

The microalgae observation apparatus according to the first embodiment enables screening for determining culture conditions or other environmental conditions helping produce microalgae containing a large amount of lipid. A method for screening microalgae culture conditions may include, for example: introducing a plurality of fluids into a flow cell 40, the fluids each containing microalgae being cultured under a condition different from the microalgae in the other fluids; irradiating the flow cell 40 with excitation light; detecting autofluorescence emitted from lipid of each of the microalgae irradiated with the excitation light; allowing the recording section 301 to time-sequentially record the intensity of the detected lipid autofluorescence and the number of signals of lipid autofluorescence emitted within a unit time in the recording device 351 for each microalgae culture condition; allowing the quantitative determination section 304 to calculate the amount and the concentration of lipid for each microalgae culture condition from the volume of the corresponding fluid that has passed through the flow cell 40 within a unit time, the intensity of lipid autofluorescence detected within the unit time, and the number of detected signals of lipid autofluorescence emitted within the unit time; and selecting the microalgae culture condition in which the amount and the concentration of the lipid each exceed a predetermined criterion value. The predetermined criterion values are appropriately set.

The screening of microalgae and the screening of culture conditions may be combined.

The microalgae observation apparatus according to the first embodiment enables the monitoring of the environment of the source of fluid containing microalgae. Examples of the source of the fluid containing microalgae include rivers, ponds, sea, and water treatment plants. The method for monitoring environment includes: introducing a fluid containing microalgae into a flow cell 40; irradiating the flow cell 40 with excitation light; detecting lipid autofluorescence emitted from lipid of each of the microalgae irradiated with the excitation light; detecting chloroplast autofluorescence emitted from chloroplasts of each of the microalgae irradiated with the excitation light; detecting scattered light from each of the microalgae; estimating the state of the microalgae from the intensity of the detected lipid autofluorescence, the number of detected signals of lipid autofluorescence emitted within a unit time, the intensity of the detected chloroplast autofluorescence, the number of detected signals of chloroplast autofluorescence emitted within the unit time, the intensity of the detected scattered light, and the number of detected signals of light scattered within the unit time; and estimating the environment of the source of the fluid containing the microalgae from a result of the estimation of the state of the microalgae.

### Second Embodiment

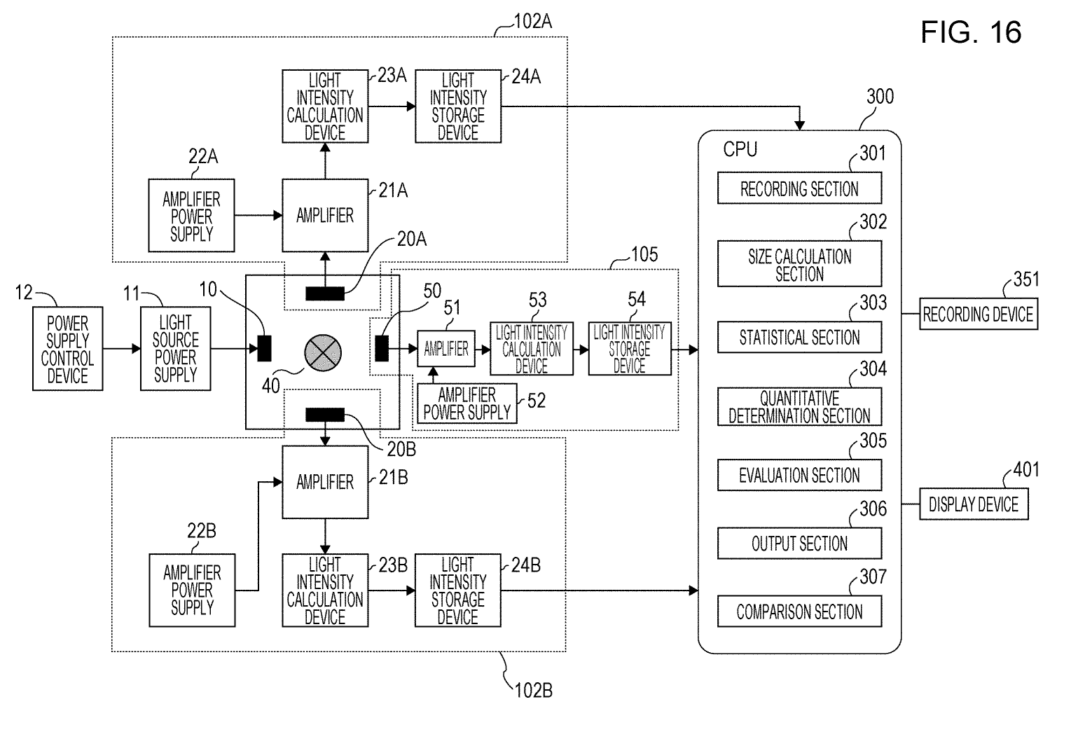

As shown in FIG. 16, the CPU 300 of a microalgae observation apparatus according to a second embodiment further includes a comparison section 307 that compares the intensities of simultaneously detected scattered light, lipid autofluorescence and chloroplast autofluorescence.

The comparison section 307 may calculate, for example, the ratio of the intensity of autofluorescence emitted from the lipid of microalgae to the intensity of scattered light. The comparison section 307 may normalize the intensity value of scattered light into 100 and calculate the ratio of the intensity of autofluorescence emitted from the lipid of microalgae to the normalized intensity of scatted light.

The comparison section 307 may calculate, for example, the ratio of the intensity of autofluorescence emitted from the chloroplasts of microalgae to the intensity of scattered light. The comparison section 307 may calculate the ratio of the intensity of autofluorescence emitted from the chloroplasts of microalgae to the normalized intensity of scattered light.

The comparison section 307 may compare the sizes of microalgae, lipid and chloroplasts that are calculated by the size calculation section 302.

In the second embodiment, the evaluation section 305 may estimate the state of the microalgae from the results of comparison among the intensity of light scattered from the microalgae, the intensity of autofluorescence emitted from the lipid, and the intensity of autofluorescence emitted from the chloroplasts.

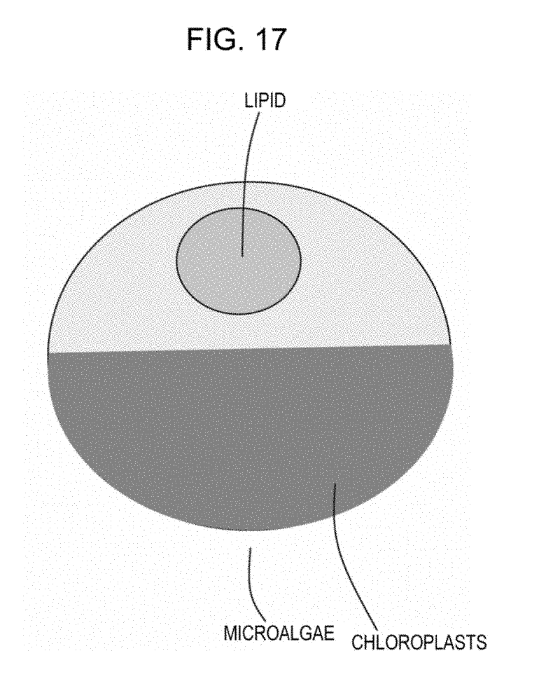

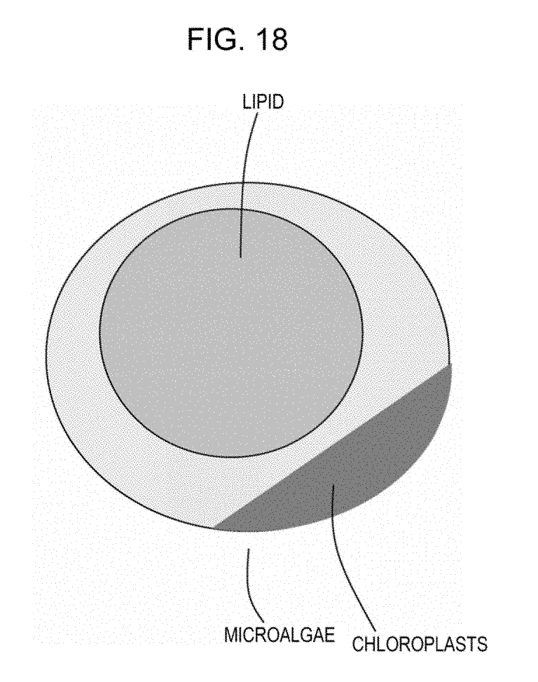

For example, if the distribution of the ratio of the intensity of autofluorescence emitted from the lipid of each microalga to the intensity of scattered light from the microalga is smaller than a predetermined criterion value, it is estimated that the proportion of the lipid in the microalga is smaller as shown in FIG. 17. Also, if the distribution of the ratio of the intensity of autofluorescence emitted from the lipid of each microalga to the intensity of scattered light from the microalga is larger than a predetermined criterion value, it is estimated that the proportion of the lipid in the microalga is larger as shown in FIG. 18.

Furthermore, for example, if the distribution of the ratio of the intensity of autofluorescence emitted from the chloroplasts of each microalga to the intensity of scattered light from the microalga is smaller than a predetermined criterion value, it is estimated that the proportion of the chloroplasts in the microalga is smaller as shown in FIG. 18. Also, if the distribution of the ratio of the intensity of autofluorescence emitted from the chloroplasts of each microalga to the intensity of scattered light from the microalga is larger than a predetermined criterion value, it is estimated that the proportion of the chloroplasts in the microalga is larger as shown in FIG. 17.

The microalgae observation apparatus according to the second embodiment enables the proportion of the size of lipid to the size of microalgae to be obtained by comparing the intensities of scatted light and lipid autofluorescence.

For example, for extracting lipid from microalgae, when the distribution of the ratio of the intensity of autofluorescence emitted from the lipid of microalgae to the intensity of scattered light from the microalgae exceeds a predetermined criterion value, it is determined that this is the timing at which the lipid is to be extracted from the microalgae, and the lipid may be extracted from the microalgae. Alternatively, when the distribution of the ratio of the intensity of autofluorescence emitted from the lipid of microalgae to the intensity of autofluorescence from the chloroplasts of the microalgae exceeds a predetermined criterion value, it is determined that this is the timing at which the lipid is to be extracted from the microalgae, and the lipid may be extracted from the microalgae.

In a screening of microalgae, the kind of microalgae may be selected in which the ratio of the intensity of autofluorescence from the lipid to the intensity of scattered light exceeds a predetermined criterion value. Alternatively, the kind of microalgae may be selected in which the ratio of the intensity of autofluorescence from the lipid to the intensity of autofluorescence from the chloroplasts exceeds a predetermined criterion value.

Also, in a screening of microalgae culture conditions, the culture condition may be selected where the ratio of the intensity of autofluorescence from the lipid to the intensity of scattered light exceeds a predetermined criterion value. Alternatively, the microalgae culture condition may be selected where the ratio of the intensity of autofluorescence from the lipid to the intensity of autofluorescence from the chloroplasts exceeds a predetermined criterion value.

### Reference Example 1

A part of Chlorella vulgaris Beijerinck (NIES-2170) was distributed from the National Institute for Environmental Studies, Microorganism Strain Storage Facility (Japan). The chlorella was cultured in liquid C culture medium in a thermostatic chamber of 25° C. A test tube containing the chlorella and the liquid C culture medium was shaken at 100 rpm during culture. In the thermostatic chamber during the culture, 10-hour lighting of daylight color fluorescent light and 14-hours non-lighting were repeated according to the recommendation of the distribution institute for the culture conditions.

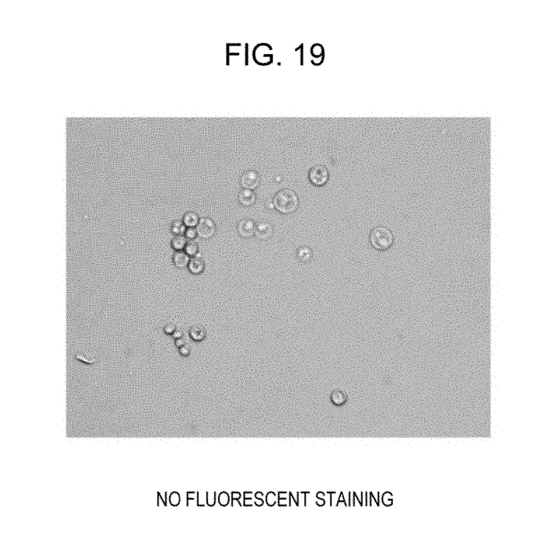

Onto a slide glass was dropped 10 μL of liquid C culture medium containing cultured chlorella not stained with a fluorescent dye, and the dropped sample was covered with a cover glass. Subsequently, the transmission micrograph, shown in FIG. 19, of the chlorella not stained with a fluorescent dye was taken under a transmission microscope mounted on UIS manufactured by Olympus.

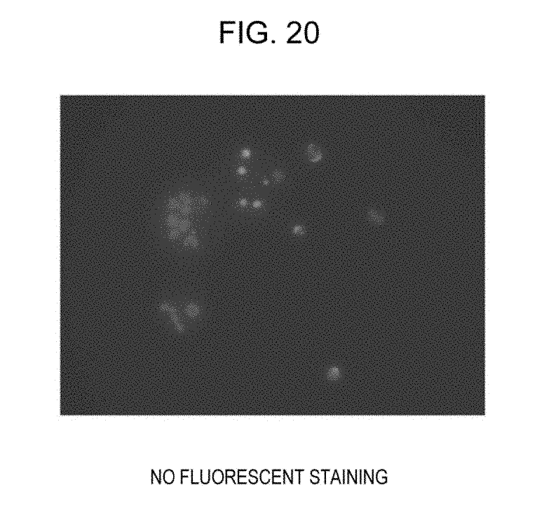

Then, the fluorescence micrograph, shown in FIG. 20, of the chlorella not stained with a fluorescent dye was taken under the same microscope without moving the slide glass. More specifically, wideband (WIB) excitation light was emitted from an excitation light source. The light was filtered into light in a wavelength range of 460 nm to 495 nm through a bandpass filter (BP 460-495), and the chlorella not stained with a fluorescent dye was irradiated with this excitation light through an objective lens. The autofluorescence emitted from the chlorella irradiated with the excitation light and not stained with a fluorescent dye was photographed with a camera through an objective lens and an absorption filter (BA510IF) that absorbs light having a wavelength of less than 510 nm and transmits light having a wavelength of 510 nm or more. The time of irradiation with the excitation light (time of chlorella exposure) was 1.0 second. No neutral density (ND) filter was not used for the excitation light.

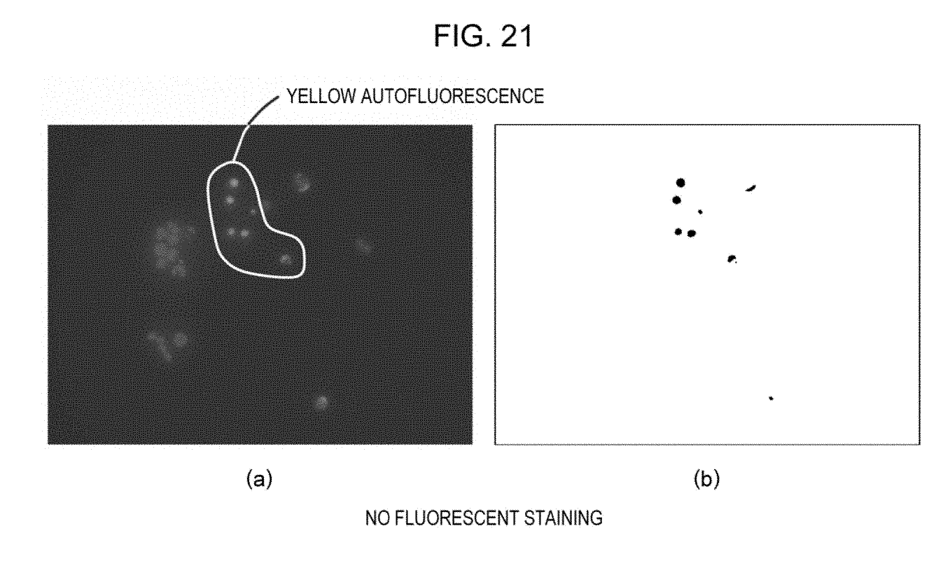

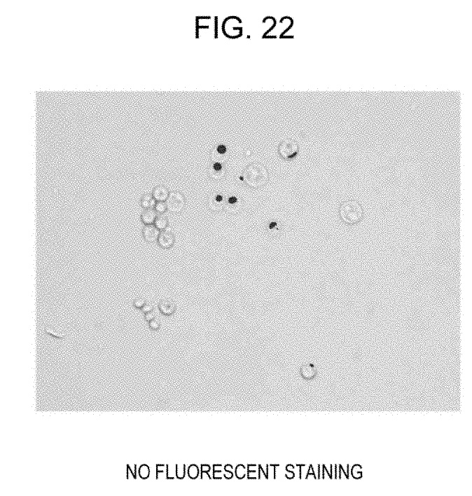

In the fluorescence microscope of chlorella shown in FIG. 21(a), mainly yellow autofluorescence was observed in the region surrounded by a line. In the other region, mainly red autofluorescence was observed. A yellow autofluorescence-extracted image as shown in FIG. 21(b), in which the portions of the chlorella fluorescence micrograph from which yellow autofluorescence was emitted were extracted as black portions while the other portion was converted into a white portion, was formed by using an image processing software program (ImagePro). When the yellow autofluorescence-extracted image shown in FIG. 21(b) was superimposed over the transmission micrograph shown in FIG. 19, the shapes of intracellular tissues observed in the transmission micrograph correspond to the shapes of the portions from which the yellow autofluorescence was emitted, as shown in FIG. 22.

### Reference Example 2

A 1 mg/mL fluorescent reagent solution was prepared by diluting BODIPY (registered trademark) 493/503, which is a lipid labeling fluorescent dye having a peak wavelength of 503 nm, with ethanol. Then, 0.1 μL of the fluorescent reagent solution was added into 100 μL of liquid C culture medium containing chlorella cultured in the same manner as in Reference Example 1 to stain the chlorella with BODIPY (registered trademark).

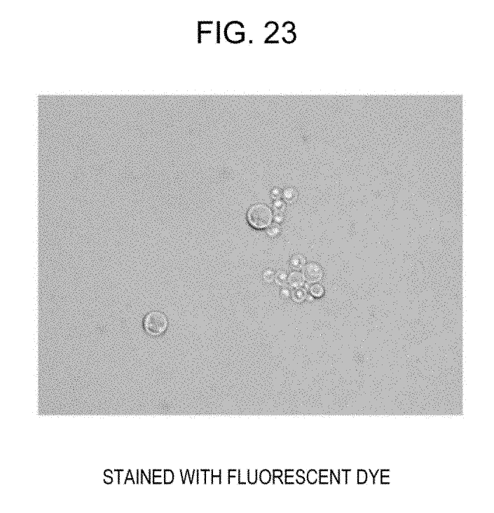

On the same day as the microscope observation in Reference Example 1, 10 μL of liquid C culture medium containing the chlorella stained with BODIPY (registered trademark) was dropped onto a slid glass and covered with a cover glass. Subsequently, the transmission micrograph, shown in FIG. 23, of the chlorella stained with BODIPY (registered trademark) was taken under the transmission microscope mounted on UIS manufactured by Olympus.

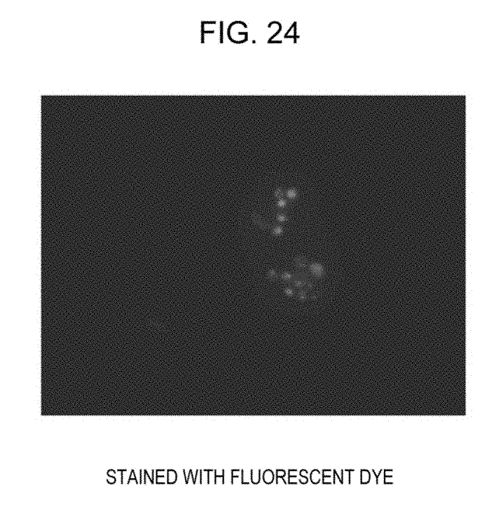

Then, the fluorescence micrograph, shown in FIG. 24, of the chlorella stained with BODIPY (registered trademark) was taken under the same microscope without moving the slide glass. More specifically, wideband (WIB) excitation light was emitted and filtered into light in a wavelength range of 460 nm to 495 nm through a bandpass filter (BP 460-495), and the chlorella stained with BODIPY (registered trademark) was irradiated with this excitation light through an objective lens. The fluorescence emitted from the chlorella irradiated with the excitation light and stained with BODIPY (registered trademark) was photographed with a camera through an objective lens and an absorption filter (BA510IF) that absorbs light having a wavelength of less than 510 nm and transmits light having a wavelength of 510 nm or more. The time of irradiation with the excitation light (time of chlorella exposure) was 0.5 second. In this Example, an ND filter having an average transmittance (Tav) of 25% was used for the excitation light.

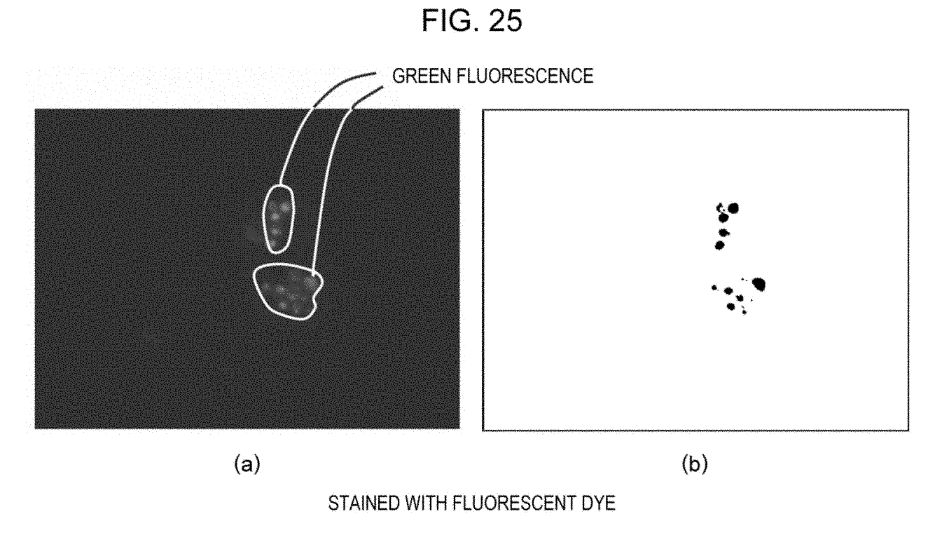

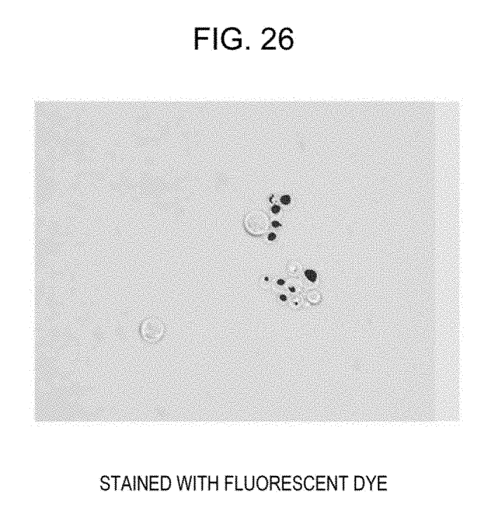

In the fluorescence microscope of chlorella shown in FIG. 25(a), mainly green fluorescence was observed in the regions surrounded by respective lines. In the other region, mainly red fluorescence was observed. A green fluorescence-extracted image as shown in FIG. 25(b), in which the portions of the chlorella fluorescence micrograph from which green fluorescence was emitted were extracted as black portions while the other portion was converted into a white portion, was formed by using an image processing software program (ImagePro). When the green fluorescence-extracted image shown in FIG. 25(b) was superimposed over the transmission micrograph shown in FIG. 23, the shapes of intracellular tissues observed in the transmission micrograph correspond to the shapes of the portions from which the green fluorescence was emitted, as shown in FIG. 26.

Also, the shapes of the portions in which fluorescence from the chlorella stained with a known lipid labeling reagent BODIPY (registered trademark) was observed were similar to the shapes of the portions in which yellow autofluorescence from the chlorella not stained with a fluorescent dye shown in FIG. 22 was observed. This suggests that the lipid in the chlorella emits autofluorescence that is observed as yellow emission when a bandpass filter (BP 460-495) and an absorption filter (BA510IF) are used.

### Reference Example 3

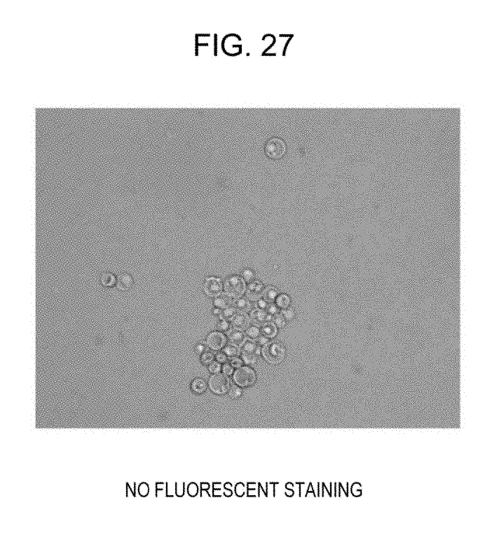

Onto a slide glass was dropped 10 μL of liquid C culture medium containing chlorella cultured in the same manner as in Reference Example 1 and not stained with a fluorescent dye, and the dropped sample was covered with a cover glass. Subsequently, the transmission micrograph, shown in FIG. 27, of the chlorella not stained with a fluorescent dye was taken under a transmission microscope mounted on UIS manufactured by Olympus.

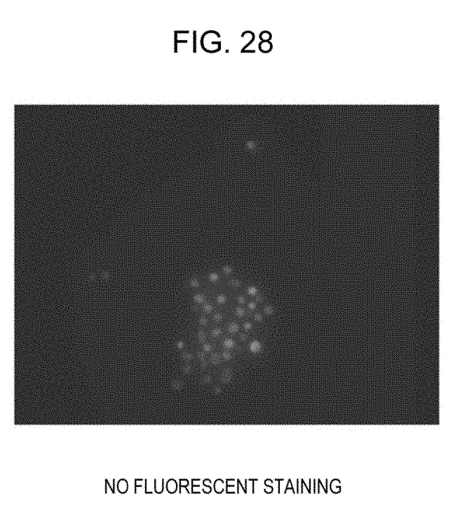

Then, the fluorescence micrograph, shown in FIG. 28, of the chlorella not stained with a fluorescent dye was taken under the same microscope without moving the slide glass. The photograph was taken under the same conditions as the photograph shown in FIG. 20 in Reference Example 1.

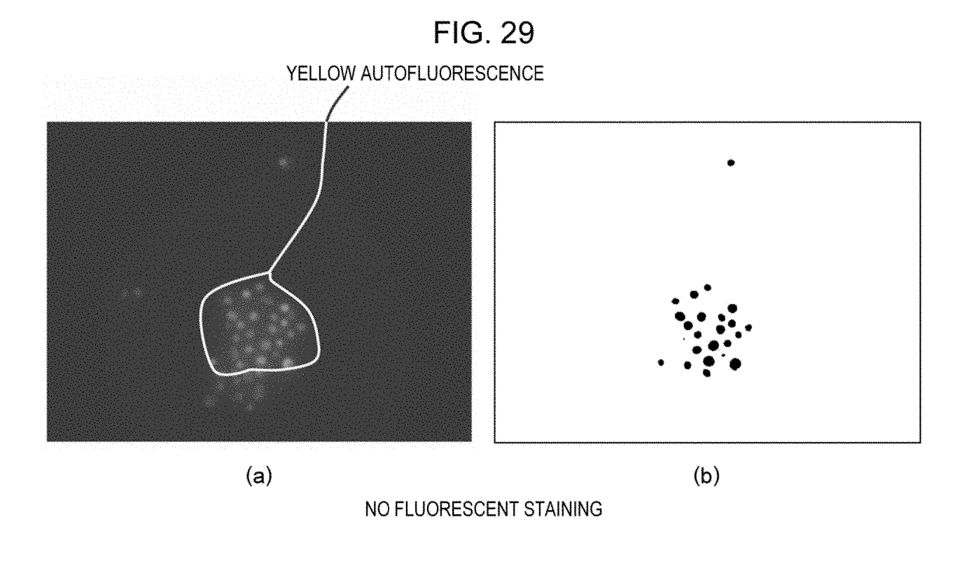

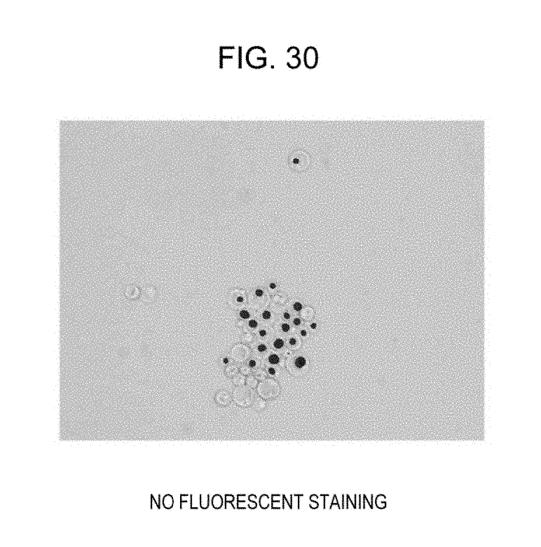

In the fluorescence microscope of chlorella shown in FIG. 29(a), mainly yellow autofluorescence was observed in the region surrounded by a line. In the other region, mainly red autofluorescence was observed. An autofluorescence-extracted image as shown in FIG. 29(b), in which the portions of the chlorella fluorescence micrograph from which yellow autofluorescence was emitted were extracted as black portions while the other portion was converted into a white portion, was formed by using an image processing software program (ImagePro). When the yellow autofluorescence-extracted image shown in FIG. 29(b) was superimposed over the transmission micrograph shown in FIG. 27, the shapes of intracellular tissues observed in the transmission micrograph correspond to the shapes of the portions from which the yellow autofluorescence was emitted, as shown in FIG. 30.

### Reference Example 4

A 1 mg/mL fluorescent reagent solution was prepared by diluting Nile Red, which is a lipid labeling fluorescent dye having a peak wavelength of 637 nm, with acetone. Then, 1.0 μL of the fluorescent reagent solution was added into 200 μL of liquid C culture medium containing chlorella cultured in the same manner as in Reference Example 3 to stain the chlorella with Nile Red.

On the same day as the microscope observation in Reference Example 3, 10 μL of liquid C culture medium containing the chlorella stained with Nile Red was dropped onto a slid glass and covered with a cover glass.

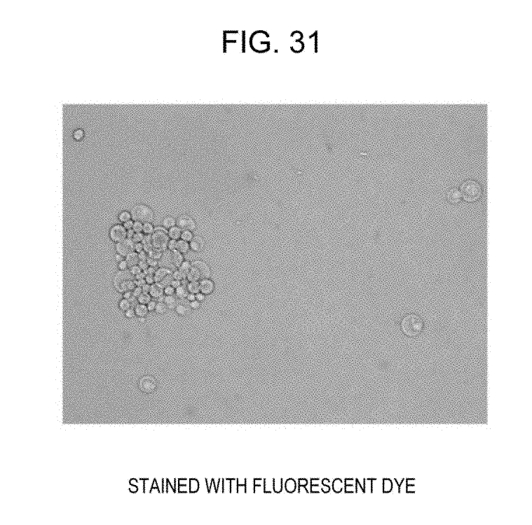

Subsequently, the transmission micrograph, shown in FIG. 31, of the chlorella stained with Nile Red was taken under the transmission microscope mounted on UIS manufactured by Olympus.

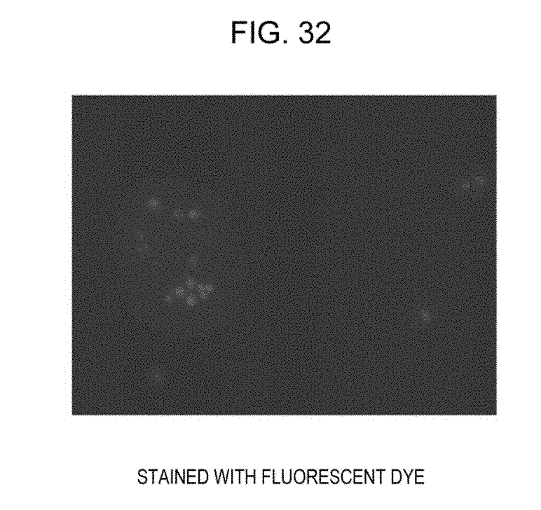

Then, the fluorescence micrograph, shown in FIG. 32, of the chlorella stained with Nile Red was taken under the same microscope without moving the slide glass. More specifically, wideband (WIB) excitation light was emitted and filtered into light in a wavelength range of 530 nm to 550 nm through a bandpass filter (BP 530-550), and the chlorella stained with Nile Red was irradiated with this excitation light through an objective lens. The fluorescence emitted from the chlorella irradiated with the excitation light and stained with Nile Red was photographed with a camera through an objective lens and an absorption filter (BA575IF) that absorbs light having a wavelength of less than 575 nm and transmits light having a wavelength of 575 nm or more. The time of irradiation with the excitation light (time of chlorella exposure) was 1.0 second. In this Example, an ND filter having an average transmittance (Tav) of 25% and an ND filter having an average transmittance (Tav) of 6% were used for the excitation light.

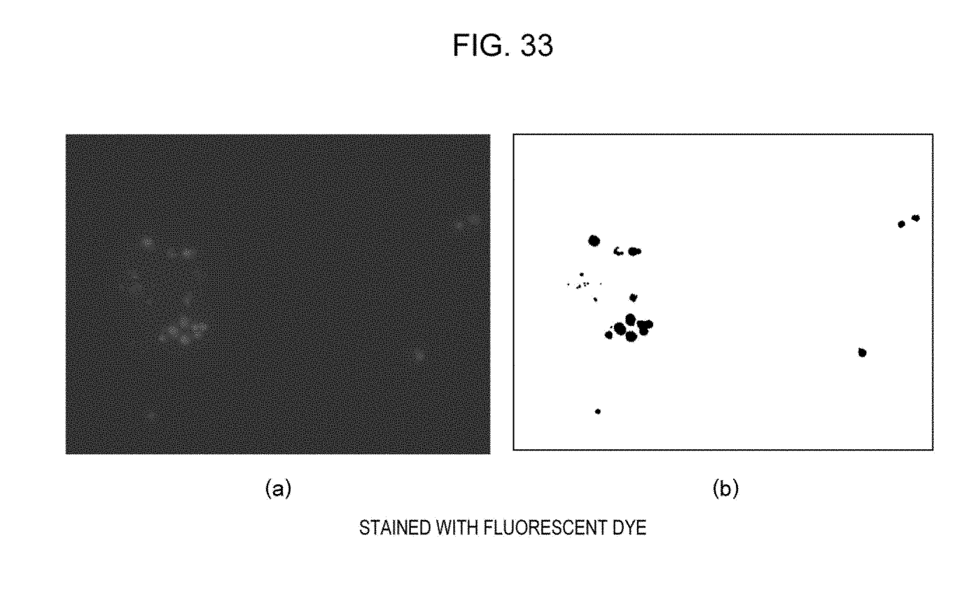

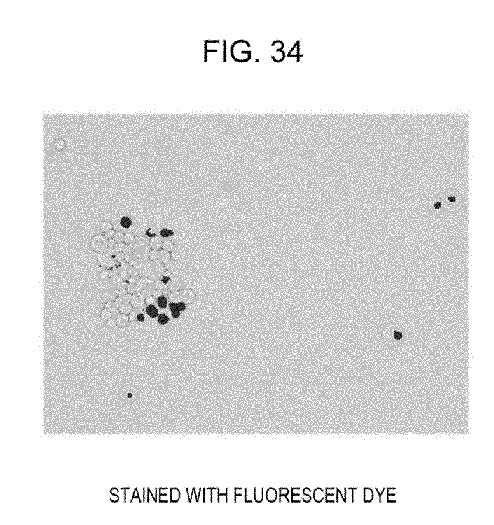

In the fluorescence microscope of chlorella shown in FIG. 33(a), mainly red fluorescence was observed. A red fluorescence-extracted image as shown in FIG. 33(b), in which the portions of the chlorella fluorescence micrograph from which red fluorescence was emitted were extracted as black portions while the other portion was converted into a white portion, was formed by using an image processing software program (ImagePro). When the red fluorescence-extracted image shown in FIG. 33(b) was superimposed over the transmission micrograph shown in FIG. 31, the shapes of intracellular tissues observed in the transmission micrograph correspond to the shapes of the portions from which the red fluorescence was emitted, as shown in FIG. 34.

Also, the shapes of the portions in which fluorescence from the chlorella stained with a known lipid labeling reagent Nile Red was observed were similar to the shapes of the portions in which yellow autofluorescence from the chlorella not stained with a fluorescent dye shown in FIG. 30 was observed through a bandpass filter (BP 460-495) and an absorption filter (BA510IF).

### REFERENCE SIGNS LIST

10 excitation light source
11 light source driving power supply
12 power supply control device
20A first light-receiving element
20B second light-receiving element
21A, 21B, 51 amplifier
22A, 22B, 52 amplifier power supply
23A, 23B, 53 light intensity calculation device
24A, 24B, 54 light intensity storage device
40 flow cell
50 scattered light-receiving element
100 culture vessel
102A first fluorescence detector
102B second fluorescence detector
105 scattered light detector
301 recording section
302 size calculation section
303 statistical section
304 quantitative determination section
305 evaluation section
306 output section
307 comparison section
351 recording device
401 display device

1. A microalgae monitoring apparatus comprising:
a flow cell into which a fluid containing microalgae is introduced;
an excitation light source configured to irradiate the flow cell with excitation light;
a fluorescence detector configured to detect lipid autofluorescence emitted from lipid of each of the microalgae irradiated with the excitation light;
a scattered light detector configured to detect light scattered from each of the microalgae; and
a processing unit configured to time-sequentially record the intensities of the detected lipid autofluorescence and scattered light.

2. The microalgae monitoring apparatus according to claim 1, wherein the lipid autofluorescence is yellow.

3. The microalgae monitoring apparatus according to claim 1, wherein the processing unit calculates the size of the microalgae from the intensity of the scattered light and calculates the size of the lipid from the intensity of the lipid autofluorescence.

4. The microalgae monitoring apparatus according to claim 3, wherein the processing unit calculates the distributions of the size of the microalgae measured within a unit time and the size of the lipid measured within the unit time.

5. The microalgae monitoring apparatus according to claim 4, wherein the unit time for calculating the distributions is shifted on a time series.

6. The microalgae monitoring apparatus according to claim 3, wherein the processing unit records changes with time in size of the microalgae and in size of the lipid.

7. The microalgae monitoring apparatus according to claim 1, wherein the processing unit
calculates the amount and the concentration of the microalgae from the volume of the fluid that has passed through the flow cell within a unit time, the intensity of light scattered from the microalgae within the unit time, and the number of detected signals of the light scattered from the microalgae within the unit time, and
calculates the amount and the concentration of the lipid from the volume of the fluid that has passed through the flow cell within a unit time, the intensity of lipid autofluorescence detected within the unit time, and the number of detected signals of lipid autofluorescence emitted within the unit time.

8. The microalgae monitoring apparatus according to claim 7, wherein the processing unit records changes with time in amount and concentration of the microalgae and in amount and concentration of the lipid.

9. The microalgae monitoring apparatus according to claim 1, further comprising a fluorescence detector configured to detect chloroplast autofluorescence emitted from chloroplasts of each of the microalgae.

10. The microalgae monitoring apparatus according to claim 9, wherein the processing unit calculates the size of the microalgae from the intensity of the scattered light, calculates the size of the lipid from the intensity of the lipid autofluorescence, and calculates the size of the chloroplasts from the intensity of the chloroplast autofluorescence.

11. The microalgae monitoring apparatus according to claim 10, wherein the processing unit calculates the distributions of the size of the microalgae measured within a unit time, the size of the lipid measured within the unit time, and the size of the chloroplasts measured within the unit time.

12. The microalgae monitoring apparatus according to claim 10, wherein the processing unit records changes with time in size of the microalgae, in size of the lipid, and in size of the chloroplasts.

13. The microalgae monitoring apparatus according to claim 9, wherein the processing unit
calculates the amount and the concentration of the microalgae from the volume of the fluid that has passed through the flow cell within a unit time, the intensity of light scattered from the microalgae within the unit time, and the number of detected signals of the light scattered from the microalgae within the unit time,
calculates the amount and the concentration of the lipid from the volume of the fluid that has passed through the flow cell within the unit time, the intensity of lipid autofluorescence detected within the unit time, and the number of detected signals of lipid autofluorescence emitted within the unit time, and
calculates the amount and the concentration of the chloroplasts from the volume of the fluid that has passed through the flow cell within the unit time, the intensity of chloroplast autofluorescence detected within the unit time, and the number of detected signals of chloroplast autofluorescence emitted within the unit time.

14. The microalgae monitoring apparatus according to claim 13, wherein the processing unit records changes with time in amount and concentration of the microalgae, in amount and concentration of the lipid, and in amount and concentration of the chloroplasts.

15. The microalgae monitoring apparatus according to claim 3, further comprising a display device capable of displaying calculation results.

16. A method for monitoring microalgae, the method comprising:
introducing a fluid containing microalgae into a flow cell;
irradiating the flow cell with excitation light;
detecting lipid autofluorescence emitted from lipid of each of the microalgae irradiated with the excitation light;
detecting light scattered from each of the microalgae; and
time-sequentially recording the intensities of the detected lipid autofluorescence and scattered light.

17. A method for determining a timing at which a microalgae culture is to be finished, the method comprising:
introducing a fluid containing microalgae into a flow cell;
irradiating the flow cell with excitation light;
detecting lipid autofluorescence emitted from lipid of each of the microalgae irradiated with the excitation light;
time-sequentially recording the intensity of the detected lipid autofluorescence;
calculating the amount and the concentration of the lipid from the volume of the fluid that has passed through the flow cell within a unit time, the intensity of lipid autofluorescence detected within the unit time, and the number of detected signals of lipid autofluorescence emitted within the unit time, and
determining, when the amount and the concentration of the lipid each exceed a predetermined criterion value, that this time is the timing at which the microalgae culture is to be finished.

18. A method for screening microalgae, the method comprising:
introducing each of a plurality of fluids into a flow cell, the fluids each containing a different kind of microalgae from the microalgae in the other fluids;
irradiating the flow cell with excitation light;
detecting lipid autofluorescence emitted from lipid of each of the microalgae irradiated with the excitation light;
time-sequentially recording the intensity of the detected lipid autofluorescence for each kind of microalgae;
calculating the amount and the concentration of the lipid for each kind of microalgae from the volume of the corresponding fluid that has passed through the flow cell within a unit time, the intensity of lipid autofluorescence detected within the unit time, and the number of detected signals of lipid autofluorescence emitted within the unit time, and
selecting the kind of microalgae in which the amount and the concentration of the lipid each exceed a predetermined criterion value.

19. A method for screening microalgae culture conditions, the method comprising:
introducing a plurality of fluids into a flow cell, the fluids each containing microalgae being cultured under a culture condition different from the microalgae in the other fluids;
irradiating the flow cell with excitation light;
detecting lipid autofluorescence emitted from lipid of each of the microalgae irradiated with the excitation light;
time-sequentially recording the intensity of the detected lipid autofluorescence for each microalgae culture condition;
calculating the amount and the concentration of the lipid for each microalgae culture condition from the volume of the corresponding fluid that has passed through the flow cell within a unit time, the intensity of lipid autofluorescence detected within the unit time, and the number of detected signals of lipid autofluorescence emitted within the unit time, and
selecting the culture condition in which the amount and the concentration of the lipid each exceed a predetermined criterion value.

20. A method for monitoring environment, the method comprising:
introducing a fluid containing microalgae into a flow cell;
irradiating the flow cell with excitation light;
detecting lipid autofluorescence emitted from lipid of each of the microalgae irradiated with the excitation light;
detecting chloroplast autofluorescence emitted from chloroplasts of each of the microalgae irradiated with the excitation light;
detecting light scattered from each of the microalgae;
estimating the state of the microalgae from the intensity of the detected lipid autofluorescence, the number of detected signals of lipid autofluorescence emitted within a unit time, the intensity of the detected chloroplast autofluorescence, the number of detected signals of chloroplast autofluorescence emitted within the unit time, and the intensity of the detected scattered light, and the number of detected signals of light scattered within the unit time; and
estimating the environment of the source of the fluid containing the microalgae from a result of the estimation of the state of the microalgae.
